(19) United States
(12) Patent Application Publication
(10) Pub. No.: US 2015/0050371 A1
(43) Pub. Date: Feb. 19, 2015

(54) Extract Formulations of Rhodamnia Cinerea And Uses Thereof

(71) Applicants: Matthias Gehling, Leichlingen (DE); Bärbel Köpcke, Dortmund (DE); Thomas Küper, Reken (DE); Kathrin Reinhardt, Dortmund (DE); Peter Reinemer, Munchen (DE); Annie George, Puchong (MY); Sasikala Chinnappan, Puchong (MY); Mazria Haslina Md Akir, Padang Besar (MY)

(72) Inventors: Matthias Gehling, Leichlingen (DE); Bärbel Köpcke, Dortmund (DE); Thomas Küper, Reken (DE); Kathrin Reinhardt, Dortmund (DE); Peter Reinemer, Munchen (DE); Annie George, Puchong (MY); Sasikala Chinnappan, Puchong (MY); Mazria Haslina Md Akir, Padang Besar (MY)

(73) Assignee: BIOTROPICS MALAYSIA BERHAD, Kuala Lumpur (MY)

(21) Appl. No.: 14/382,237

(22) PCT Filed: Mar. 9, 2012
(86) PCT No.: PCT/MY2012/000048

§ 371 Date: Aug. 29, 2014

(51) Int. Cl. A61K 36/61; A23L 1/30; A61K 45/06

(52) U.S. Cl. CPC A61K 36/61; A61K 45/06; A23L 1/3002; A23V 2002/00
USPC 424/773

(57) ABSTRACT

The present invention primarily relates to the use of certain extract formulations of Rhodamnia cinerea as defined herein as alpha-amylase inhibitors and as actives for the therapeutic (including prophylactic) treatment of a carbohydrate metabolic disorder or of a disease attendant on hyperglycemia, preferably selected from the group consisting of prediabetes, obesity, hyperlipemia, arteriosclerosis, arteriolosclerosis, atherosclerosis, diabetes, postprandial hyperglycemia, and metabolic syndrome. The present invention also relates to corresponding methods. The invention further relates to specific extract formulations obtainable from Rhodamnia cinerea and to compositions, in particular orally consumable compositions, comprising an effective amount of such an extract formulation.

The present invention primarily relates to the use of certain extract formulations of Rhodamnia cinerea as defined herein as alpha-amylase inhibitors and as actives for the therapeutic (including prophylactic) treatment of a carbohydrate metabolic disorder or of a disease attendant on hyperglycemia, preferably selected from the group consisting of prediabetes, obesity, hyperlipemia, arteriosclerosis, arteriolosclerosis, atherosclerosis, diabetes, postprandial hyperglycemia, and metabolic syndrome. The present invention also relates to corresponding methods. The invention further relates to specific extract formulations obtainable from Rhodamnia cinerea and to compositions, in particular orally consumable compositions, comprising an effective amount of such an extract formulation.

Diabetes mellitus, often simply referred to as diabetes, is a group of metabolic diseases in which a person has high blood sugar, either because the body does not produce enough insulin, or because cells do not respond to the insulin that is produced. Diabetes is one of the most common metabolic disorders worldwide: more than 170 million people worldwide are affected by diabetes. Non-insulin dependent diabetes mellitus (nowadays and hereinafter referred to as type II diabetes or type 2 diabetes) is by far the most common, affecting 90 to 95% of the diabetes population. Type II diabetes results from insulin resistance, a condition in which cells fail to use insulin properly, sometimes combined with insulin deficiency.

Hydrolysis of carbohydrates mediated by enzymes, in particular by α-amylase and α-glucosidases, followed by glucose uptake results in sudden rise in blood glucose levels, causing hyperglycemia in type II diabetes patients. Hyperglycemia is particularly pronounced and long-lasting in diabetics.

Type II diabetes mellitus is closely related to obesity, and may cause chronic hyperglycemia due to insulin resistance. Furthermore, type II diabetes causes complications, such as retinopathy, nephritis, cardiovascular diseases, and neurological disorders. Diet and exercise therapy are the key factors for preventing and treating type II diabetes. In dieting, controlling blood glucose levels in everyday life is especially important. Blood glucose levels are greatly affected by the saccharides (starches, glycogen, sugars, etc.) contained in food. These saccharides are decomposed by the actions of alpha-amylase and alpha-glucosidase, which are digestive enzymes (carbohydrases). Alpha-amylase is an enzyme that hydrolyzes the alpha-1,4-glucoside linkages of starches and glycogen. These enzymes are contained in the saliva and pancreatic fluid of animals, and transform starches and the like into maltose, etc., in the alimentary canal.

Diabetes increases the risk of long-term complications. The major long-term complications relate to damage to blood vessels. Diabetes clearly increases the risk of cardiovascular diseases. The main “macrovascular” diseases (related to atherosclerosis of larger arteries) are ischemic heart disease, stroke and peripheral vascular disease.

Hypertension (or high blood pressure) is a condition which occurs in the human population as a secondary symptom to various other disorders. Hypertension is often associated with disorders such as obesity, diabetes and hypertriglyceridemia.

Hypertension can also contribute to the development of atherosclerosis and coronary disease.

The “metabolic syndrome” (also called metabolic syndrome X or syndrome X) is a combination of medical disorders that, when occurring together, increase the risk of developing cardiovascular disease and diabetes. The “metabolic syndrome” is defined by various organizations referring to risk parameters which are defined by different critical values. In the context of the present text, the definition established by the International Diabetes Federation of the metabolic syndrome (2006) applies:

Central obesity (defined as waist circumference with ethnicity specific values—in case the body mass index is >30 kg/m², central obesity can be assumed and waist circumference does not need to be measured),

and any two of the following:
i) raised triglycerides: >150 mg/dL (1.7 mmol/L), or specific treatment for this lipid abnormality,
ii) reduced HDL cholesterol: <40 mg/dL (1.03 mmol/L) in males, <50 mg/dL (1.29 mmol/L) in females, or specific treatment for this lipid abnormality,
iii) raised blood pressure (BP): systolic BP>130 or diastolic BP>85 mm Hg, or treatment of previously diagnosed hypertension,
iv) raised fasting plasma glucose (FPG): FPG>100 mg/dL (5.6 mmol/L), or previously diagnosed type II diabetes.

Metabolic syndrome relates to conditions wherein generally several of the following disorders are present: type II diabetes, hypertension, central obesity (a disproportionate amount of body fat in the abdominal region), hyperlipemia (also called hyperlipidemia, i.e. elevated levels of lipids, particularly triglycerides, in the blood), heart disease, atherosclerosis. Type II diabetes in the context with the metabolic syndrome is often accompanied with hypertension.

Obesity (=adiposity) is one of the main factors in the development of cardiovascular diseases. As a side effect the levels of cholesterol, blood pressure, blood sugar and uric acid in obese people are usually higher than those of persons of normal weight.

Glucose metabolism plays important roles in the development of diabetes and obesity, and restricted glucose uptake is an effective therapeutic means for diabetes and obesity. Glucose absorbed from the small intestine is carried into the blood, and raises the blood glucose level. Therefore, to inhibit a superfluous energy supply or control blood glucose levels, in other words, to prevent or treat obesity and diabetes, it is very important to control the activity of enzymes such as alpha-amylase and alpha-glucosidase.

Hence, α-amylase and α-glucosidase inhibitors can reduce the liberation of D-glucose from dietary complex carbohydrates and glucose absorption, resulting in reduced postprandial plasma glucose levels and decrease or suppression of postprandial hyperglycemia. Slower glucose absorption into the blood and a smoothing or lowering of postprandial hyperglycemia thus result in improved glycemic control.

Alpha-amylase is an enzyme that hydrolyses alpha-bonds of large alpha-linked polysaccharides such as starch and glycogen. α-Amylase catalyzes the hydrolysis of α-(1,4) glycosidic linkages of starch components and glycogen. In mammals, α-amylase is present in both salivary and pancreatic secretions. Salivary alpha-amylase (ptyalin) breaks insoluble starches and dextrins into soluble molecules (amylodextrin, erythrodextrin, achrodextrin), and subsequently into smaller carbohydrates. Ptyalin acts on linear alpha-(1,4)-glycosidic linkages. Pancreatic alpha-amylase randomly cleaves the alpha-(1-4)-glycosidic linkages of amylose to yield dextrin, maltose, and maltotriose. For example, amylopectin and amylose are cleaved by alpha-amylase into oligosaccharides, which in turn are cleaved into maltose, which is then cleaved into alpha-D-glucose by alpha-glucosidase.

An amylase inhibitor inhibits the enzymatic degradation of starch or glycogen into maltose. The inhibition of such enzymatic degradation is beneficial in reducing amounts of bioavailable sugars, including glucose and maltose, and the concomitant deleterious conditions resulting therefrom. Alimentary hyperglycemia following starch intake can be reduced by alpha-amylase inhibitors.

Alpha-amylase and alpha-glucosidase inhibitors were proved as effective means in decreasing glucose uptake and thus offering a therapeutic or prophylactic treatment for diabetic patients.

Also alpha-Glucosidase inhibitors play a major role in managing postprandial hyperglycemia in diabetic patients. Inhibition of alpha-glucosidase enzyme activity leads to a reduction in starch hydrolysis which has beneficial effects on glycemic index control in diabetic patients. An overview of glycosidase inhibitors can be found in Glycobiology 2003, 13, 93R-104R.

Alpha-glucosidase inhibitors commercially used for lowering postprandial blood glucose levels in the treatment of type 2 diabetes are, for example, acarbose, miglitol, and voglibose.

J. Verbr. Lebensm. 2011, 6:191-195 reports that several medicinal plants were screened for their α-amylase inhibitory activity, because these plants are recommended in treating diabetes in traditional Iranian medicine. Among these, extracts of Camellia sinensis (Theaceae) leaf, Trigonella foenum-graecum (Leguminosae) seed and leaf, and Urtica dioica leaf revealed appreciable α-amylase inhibitory activities in a concentration-dependent manner.

J. Ethnopharmacol. 2006, 107(3): 449-455 discloses that extracts from six Malaysian plants were examined for alpha-amylase inhibition using an in vitro model. It was reported that an extract of Phyllantus amarus had alpha-amylase inhibitory activity.

JP 2004-256432 suggests the use of an extract of Acanthopanax sieboldianum as alpha-amylase inhibitor.

U.S. Pat. No. 7,037,536 proposes the use of an extract of guava leaves as alpha-amylase inhibitor.

U.S. Pat. No. 5,643,874, US 2005/0208161 A1, US 2007/0202205 A1, and US 2007/0009615 A1 disclose different alpha-amylase and/or alpha-glucosidase inhibitors and corresponding medical or food compositions.

U.S. Pat. No. 5,468,734 proposes several monosaccharides as inhibitors of glucosidases, in particular of sucrase and maltase, such as L-arabinose, L-fucose, xylose, D-ribose, and the like.

Some naturally-occurring substances (isolated from Salacia reticulata roots and stems) like salacinol and kolatanol have been proposed as glucosidase inhibitors useful in the treatment of diabetes (see U.S. Pat. No. 6,455,573).

U.S. Pat. No. 5,021,427 relates to different naturally occurring alpha-glucosidase inhibitors like castanospermine, swainsonine, australine, DMDP and the like.

Int. J. Mol. Sci. 2011, 12, 1359-1370 discloses in vitro and in vivo anti-hyperglycemic effects based on alpha-glucosidase and alpha-amylase inhibitors of certain extracts from Omija (Schizandra chinensis) fruit.

Different traditional medicines have preventive and therapeutic effects in diabetes. To date, over 400 traditional plant treatments for diabetes have been reported, although only a small number of these have received scientific and medical evaluation. A review on natural alpha-glucosidase inhibitors useful for management of diabetes mellitus are given in Int. J. Biochem. Res. 2011, 2, 374-380 and Phcog. Rev. [serial online] 2011; 5:19-29.

The primary object of the present invention was to identify alternative alpha-amylase inhibitors or compositions useful as alpha-amylase inhibitors, in particular for the therapeutic (including prophylactic) treatment of a carbohydrate metabolic disorder or of a disease attendant on hyperglycemia. Additionally, said alpha-amylase inhibitors should preferably be naturally occurring.

It has now been found that this primary object can be achieved by using certain extract formulations obtainable from Rhodamnia cinerea.

In a first aspect, the present invention relates to a method for producing an extract formulation of Rhodamnia cinerea comprising or consisting of the following steps:
(i) providing plant material from Rhodamnia cinerea,
(i-a) optionally drying the plant material provided in step (i),
(ii) extracting the plant material provided in step (i) or (i-a) with an extractant essentially consisting or consisting of water or a mixture essentially consisting or consisting of an alcohol having 1 to 3 carbon atoms and water,
(iii) optionally mixing the extract obtained in step (ii) with one or more solid carrier substances, preferably one or more solid carrier substances selected from the group consisting of maltodextrins, silica, talc, lactose, sorbitol, mannitol, dextrose, sucrose, starches, gums, orally consumable calcium salts, orally consumable stearate salts, alginates, tragacanth, gelatins, cellulose and cellulose derivatives, polyvinylpyrrolidones, and propylhydroxybenzoates,
(iv) drying the extract obtained in step (ii) or the mixture obtained in step (iii), preferably by spray-drying or freeze-drying, preferably drying until the total amount of water and alcohols having 1 to 3 carbon atoms is below 15 wt. %, preferably below 10 wt. %, more preferably below 5 wt. %, most preferably below 3 wt. %, based on the total weight of the extract formulation.

Surprisingly, it now has been found that the extract formulations (as defined herein) can inhibit alpha-amylase, i.e. partially or fully reduce the activity of alpha-amylase, in particular of salivary and/or pancreatic alpha-amylase.

Alimentary hyperglycemia and hyperinsulinemia following starch intake can be reduced by the alpha-amylase inhibitors according to the invention. This action is dose-dependent. The alpha-amylase inhibiting extract formulations according to the invention can therefore be used for the therapeutic or prophylactic treatment of a carbohydrate metabolic disorder or of a disease attendant on hyperglycemia, preferably prediabetes, obesity, hyperlipemia, arteriosclerosis, arteriolosclerosis, atherosclerosis, diabetes, postprandial hyperglycemia, and/or treatment of metabolic syndrome. Particularly preferably the extract formulations according to the invention are used as a therapeutic agent for prediabetes, diabetes, postprandial hyperglycemia, atherosclerosis, obesity (adiposity) and/or treatment of metabolic syndrome, and as food supplement in various forms. Administration prior to or at the start of meal is advisable for this purpose. The overall daily dosage should be based on the weight of the patient and the individual requirements, and thus may deviate to some extent from the daily dosages indicated hereinbelow.

The extract formulations according to the present invention were shown in own in vitro and in vivo experiments to be potent inhibitors of (in particular pancreatic) alpha-amylase.

There is no indication hitherto that the extract formulations according to the present invention are suitable for the inhibition of alpha-amylase. The extract formulations according to the present invention by inhibiting alpha-amylase influence glycemia (i.e. the blood sugar level and/or preventing a high blood sugar level) resulting in improved glycemic control, in particular by lowering postprandial blood glucose concentration.

Therefore, extract formulations according to the present invention are suitable treatment of a disease attendant on hyperglycemia or of a carbohydrate metabolic disorder, preferably prediabetes, obesity, hyperlipemia, in particular carbohydrate-induced hyperlipemia (i.e. elevated blood lipids, particularly triglycerides, after carbohydrate ingestion; sometimes used synonymously with hyperlipoproteinemia type IV or V phenotypes), or the genetic disorders causing them), arteriosclerosis, arteriolosclerosis, atherosclerosis, and/or diabetes mellitus, in particular type 2 diabetes (type II diabetes, sometimes still called non, insulin dependent diabetes mellitus), treatment of metabolic syndrome (also called metabolic syndrome X or syndrome X), and/or treatment or prevention of postprandial hyperglycemia,

The alpha-amylase inhibiting extract formulations according to the invention are particularly suitable for oral administration. Combined use with other active substances, such as further alpha-amylase inhibitors, alpha-glucosidase inhibitors, further antidiabetic active substances, glycogen phosphorylase inhibitors, further anti-obesity agents, hypoglycemic or lipid-lowering substances, may also be advantageous and is preferred in several embodiments of the present invention.

In the context of the present invention, “essentially consists of” or “essentially consisting of” means that the total weight share is 90 wt. % or more, preferably 95 wt. % or more, more preferably 98 wt. % or more, most preferably 99 wt. % or more, based on the total amount used. For example, if plant material essentially consists leaves of Rhodamnia cinerea means that the total amount of leaves is 90 wt. % or more, preferably 95 wt. % or more, more preferably 98 wt. % or more, most preferably 99 wt. % or more, based on the total amount of plant material used.

In the context of the present invention, if a material is “essentially free of”, this means that the total weight share of other constituents is 10 wt. % or less, preferably 5 wt. % or less, more preferably 2 wt. % or less, most preferably 1 wt. % or less, based on the total amount of material used.

In the context of the present invention, a therapeutic or pharmaceutical use or method is considered as medical treatment, optionally with cosmetic (side) effects.

“Obtainable” means that a product (e.g. extract or extract formulation) may be obtained by a certain method, and preferably is obtained by said method.

Where ratios or percentages are given, these refer to the weight (e.g. percent by weight, wt. %), unless indicated otherwise. Where volume ratios (v/v) are given, these refer to the volumes at 25° C. and 1013 mbar.

“Comprising” or “including” wherever used herein is meant not to be limiting to any elements stated subsequently to such term but rather to encompass one or more further elements not specifically mentioned with or without functional importance, that is, the listed steps, elements or options need not be exhaustive. In contrast, “containing” would be used where the elements are limited to those specifically after “containing”.

By the term “extract”, either a direct extract (in liquid or preferably dried form), e.g. obtained as described below, or preferably a further enriched extract (obtainable e.g. by one or more further purification steps after extraction, e.g. chromatography, for example as described below) is meant.

By “administered” or “administering” as used herein is meant administration of a prophylactically and/or therapeutically effective amount of an extract formulation according to the present invention, to a human being in need of such treatment.

By “effective amount” as used herein is meant an amount or a dose that produces the one or more effects for which it is administered.

A “patient” or “subject” for the purposes of the present invention relates to mammals, especially human beings. Thus, extract formulations according to the present invention are applicable to both humans and mammals. In the preferred embodiment the patient is a human. The patients will be treated either in prophylactic or therapeutic intention.

The terms “dry”, “dried form”, “dry weight” and the like refer to matter (such as an extract, an extract formulation, a composition etc.) without water and without organic solvents, in particular being free of water and free of substances having a boiling point of less than 300° C. at 1013 mbar.

The terms “liquid” and “solid” refer to the state of matter, e.g. a compound, carrier or composition, at 25° C. and 1013 mbar.

The term “physiologically acceptable salt” of a compound relates to a compound in salt form which is non-toxic and orally consumable, i.e. may be safely swallowed by a mammal, preferably a human being. In particular, the term relates to a compound in which one, several or preferably all counterions (counteracting cations) are selected from the group consisting of Na⁺, K⁺, NH₄⁺, trialkylammonium NHR′₃⁺, Ca²⁺, Mg²⁺, Zn²⁺ and Al³⁺. The term “physiologically acceptable salts” also relates to and includes hydrohalides, in particular hydrochlorides, in particular in case of nitrogen containing compounds which are able to form hydrohalide salts, in particular hydrochloride salts. These preferred salts of compounds are particularly pharmaceutically or nutraceutically suitable salts.

In trialkylammonium NHR′₃⁺, preferably each R′ independently of the other radicals R′ denotes an alkyl group having 1 to 30 C-atoms, preferably having 4 to 22 C-atoms.

Particular preferred counterions in physiologically, preferably pharmaceutically or nutraceutically, acceptable salts are selected from the group consisting of Na⁺, K⁺, Ca²⁺ and Mg²⁺ and mixtures thereof.

The name “Rhodamnia cinerea” as used herein refers to Rhodamnia cinerea Jack and its synonyms.

Rhodamnia cinerea

| Scientific | |
|---|---|
| Name | Rhodamnia cinerea Jack |
| Globally | 601111-1 |
| unique | |
| identifier | |
| Class | Equisetopsida |
| Order | Myrtales |
| Plant | Myrtaceae |
| Family | |
| Reference | http://www.tropicos.org/NameDetails.aspx? |
| | nameid=50223355Malayan Miscellanies 2(7): 48 (1822) |

Synonyms of Rhodamnia cinerea Jack
http://www.nationaalherbarium.nl/sungaiwain/
Myrtaceae/Rhodamnia_cinerea.htm)
http://www.natureloveyou.sg/Plants-R.html

| | |
|---|---|
| Scientific | Rhodamnia trinervia (IPNI) |
| synonyms | Monoxora spectabilis, Myrtus globosa, Myrtus smilacifolia, |
| | Myrtus spectabilis, Rhodamnia concolor, Rhodamnia globosa, |
| | Rhodamnia nagelii, Rhodamnia spectabilis, Rhodamnia |
| | subtriflora, Rhodamnia trinervia var. concolor, Rhodamnia |
| | trinervia var. spectabilis |
| English | Silverback |
| Malay | Cherong, Siri-siri, Talinga basing (Borneo) |
| Others | Marapuyan, Memboyan |

Rhodamnia cinerea is a mid-canopy tree up to 37 m tall and 48 cm diameter at breast height. It has no stipules and the leaves are simple, triple-veined, hairy, and whitish below and they are positioned opposite. The white-pink-yellow flowers are placed in leaf axils with a diameter of ca. 8 mm. The fruits, pink-red berries, are about 7 mm in diameter and edible.

Rhodamnia cinerea can be found on Peninsular Malaysia, in Sumatra, Java, Borneo (throughout the island), Philippines, Burma and Thailand. It grows on open sites in mixed dipterocarp, keranga, coastal and submontane forests up to 1700 m altitude, on hillsides and ridges with poor sandy soils.

In Indonesia, leaves of Rhodamnia cinerea are reported to be used against aching joints (externally) and diarrhoea (oral infusions).

Additionally, a decoction of the leaves and sometimes of the roots of Rhodamnia cinerea is reported to be used after childbirth (postpartum). Methanolic extracts of the leaves of Rhodamnia cinerea showed moderate antibacterial activity against certain microorganisms like Bacillus cereus, Bacillus subtilis, and Staphylococcus aureus (Fitoterapia 2004, 75 (1), 68-73).

In summary, the biochemical and medical findings reported in the literature regarding Rhodamnia cinerea do not relate to and do not suggest the alpha-amylase inhibitory activity of extracts of Rhodamnia cinerea, in particular not the alpha-amylase inhibitory activity of the extract formulations (obtained by a method) according to the present invention.

Suitable plant materials of Rhodamnia cinerea that may be provided in step (i) of a method according to the present invention are leaves, bark, flowers, buds, fruits, stems, shoots, roots, twigs or other plant parts of Rhodamnia cinerea. Also, the whole plant of Rhodamnia may be used as plant material.

In a preferred method according to the present invention, the plant material provided in step (i) comprises fruits, flowers, leaves and/or roots of Rhodamnia cinerea, and preferably essentially consists or consists of fruits, flowers, leaves and/or roots of Rhodamnia cinerea.

In a preferred method according to the present invention, the plant material provided in step (i) comprises leaves and/or roots of Rhodamnia cinerea, and preferably essentially consists or consists of leaves, of roots or of leaves and roots of Rhodamnia cinerea.

The plant material may be used without prior treatment or after treatment, such as drying, slicing, or the like. Prior to performing the extraction step(s), the plant material is preferably comminuted, e.g. via chopping, crushing, breaking, milling or grinding or combinations thereof.

Preferably, dried (e.g. air dried) plant material of Rhodamnia cinerea is used in the extraction step (ii) of a method for producing an extract formulation according to the present invention.

Preferably, 90 wt. % or more, more preferably 95 wt. % or more, most preferably 98 wt. % or more of the total amount of plant material of Rhodamnia cinerea used in the extraction step (ii) has a particle size of less than 20 mm, more preferably of 10 mm or less, even more preferably of 6 mm or less, particularly preferably of 4 mm or less, most preferably of 2 mm or less.

The extract formulation according to the present invention or produced according to a method of the present invention may be prepared by any extraction method known in the art, however, with the proviso that certain extraction parameters are observed (as mentioned in the context of the present invention).

Auxiliary means such as (especially ultrasonic) sonication, warming/heating, stirring, may be used to allow for appropriate extraction, enrichment and purification.

The extraction can be carried out at lower or elevated or ambient temperature, e.g. in the range from 0° C. to the boiling point of the solvent or solvent mixture employed, e.g. from ambient temperature (about 20° C.) to said boiling point. The extraction may be improved by moving the solvent and/or the plant material, e.g. by stirring, and/or by ultrasonication during extraction.

Additional further processing of the (enriched) extracts used to obtain an extract formulation according to the present invention is possible, e.g. by filtering (e.g. through paper, sintered glass, charcoal (also allowing for decoloration) or silica).

In a more preferred method according to the present invention, in step (ii) the extraction is performed with an extractant
essentially consisting or consisting of water, or
a mixture essentially consisting or consisting of an alcohol having 1 to 3 carbon atoms and water, preferably a mixture of ethanol and water, wherein the total volume ratio (v/v) of said alcohol (preferably ethanol):water is in the range of 1:20 to 25:1, preferably in the range of 1:12 to 12:1, more preferably in the range of 1:6 to 10:1, even more preferably in the range of 1:5 to 5:1, particularly preferably in the range of 1:3 to 3:1, and most preferably in the range of 2:5 to 5:2.

In a preferred method according to the present invention, the extraction (step (ii)) is performed at a temperature in the range of 40 to 120° C., preferably in the range of 50 to 110° C., more preferably in the range of 60 to 100° C.

A preferred method according to the present invention for producing an extract formulation according to the present invention comprises or consists of the following steps:
(i) providing plant material from Rhodamnia cinerea,
(i-a) optionally (and preferably) drying the plant material provided in step (i),
(i-b) comminuting the plant material provided in step (i) or (i-a), preferably such that 95 wt. % or more of the total amount of the (preferably dried) plant material has a particle size of less than 20 mm, more preferably of 10 mm or less, even more preferably of 6 mm or less, particularly preferably of 4 mm or less, most preferably of 2 mm or less,
(ii) extracting the plant material provided in step (i), (i-a) or (i-b) with an extractant
   essentially consisting or consisting of water, or
   a mixture essentially consisting or consisting of ethanol and water, wherein the total volume ratio (v/v) of ethanol:water is in the range of 1:5 to 5:1, preferably in the range of 1:3 to 3:1, more preferably in the range of 2:5 to 5:2,
   wherein the extraction is performed at a temperature in the range of 50 to 110° C., preferably in the range of 60 to 100° C.,
(iii) optionally mixing the extract obtained in step (ii) with one or more solid carrier substances selected from the group consisting of maltodextrins, silica, talc, lactose, sorbitol, mannitol, starches, gums, orally consumable calcium salts, orally consumable stearate salts, alginates, tragacanth, gelatins, cellulose and cellulose derivatives, polyvinyl pyrrolidones, and propylhydroxybenzoates
(iv) drying the extract obtained in step (ii) or the mixture obtained in step (iii), preferably by spray-drying or freeze-drying, until the total amount of water and alcohols having 1 to 3 carbon atoms is below 5 wt. %, preferably below 3 wt. %, most preferably below 2 wt. %, based on the total weight of the extract formulation.

In a more preferred method according to the present invention, in step (iii) the extract obtained in step (ii) is mixed with one or more solid carrier substances selected from the group consisting of silica, talc, sorbitol, mannitol, gum acacia, calcium phosphates, calcium silicates, magnesium stearate, alginates, tragacanth, gelatins, amorphous cellulose, microcrystalline cellulose, methyl cellulose, polyvinylpyrrolidones, and propyl hydroxybenzoates,

wherein preferably the total amount of solid carrier substances [preferably of the solid carrier substances added in step (iii)] is in the range of 10 to 90 wt. %, more preferably in the range of 20 to 80 wt. %, even more preferably in the range of 30 to 75 wt. %, based on the total dry weight of the extract formulation obtained after step (iv).

The weight ratio of the total plant material to the total amount of aqueous alcoholic solvent used in the extraction preferably is in the range from 1:1 to 1:15, more preferably in the range from 1:2 to 1:10, even more preferably in the range from 1:3 to 1:8.

Preferably, the extraction of the plant material used in step (ii) of the method for producing an extract formulation according to the present invention is carried out with an extractant free of a fatty acid ester, and preferably additionally free of fatty oil (preferably free of vegetable oil) and/or free of a surfactant.

In another aspect, the present invention relates to an extract formulation in solid form obtained from plant material from Rhodamnia cinerea.

Preferably, an extract formulation according to the present invention, preferably in solid form, is essentially free of plant tissue from Rhodamnia cinerea, and particularly preferably free of plant tissue from Rhodamnia cinerea.

In another aspect, the present invention relates to an extract formulation in solid form, obtainable or obtained from plant material from Rhodamnia cinerea, preferably by a method comprising or consisting, of the steps as defined for producing a method for an extract formulation according to the present invention.

The extract formulations according to the present invention or produced according to a method of the present invention may be used as such, in the form of pharmaceutical or nutraceutical formulations (the latter term including food additives) or in the form of functional food.

“Nutraceuticals”, “Functional Food”, or “Functional Food products” (sometimes also called “Foodsceuticals”, “Medicinal Food” or “Designer Food”) for use according to the present invention are defined as food products (including beverages) suitable for human consumption—the expression comprises any fresh or processed food having a health-promoting and/or disease-preventing property beyond the basic nutritional function of supplying nutrients, including food made from functional food ingredients or fortified with health-promoting additives, especially with effects in the prophylaxis or treatment of obesity, especially allowing for body weight reduction and/or body weight maintenance, appetite suppression, the provision of satiety or similar changes in metabolism, and in which an extract formulation according to the present invention or produced according to a method of the present invention is used as an ingredient (especially additive) as health benefit agent, especially in an effective amount.

The functional food products or pharmaceutical products may be manufactured according to any suitable process, preferably admixing an extract formulation according to the present invention or produced according to a method of the present invention to a functional food product or at least one physiologically, preferably nutraceutically or pharmaceutically, acceptable carrier.

Preferably, a functional food, pharmaceutical or nutraceutical formulation comprising an extract formulation according to the present invention, can be obtained by

(a) performing an extraction from Rhodamnia cinerea plant material in accordance with a method according to the present invention, or providing an extract formulation according to the present invention,

(b) mixing the extract formulation of step (a) (as the active ingredient or one of the active ingredients) in the preparation of the functional food product with the other constituents thereof or in order to obtain a functional food, pharmaceutical or nutraceutical formulation with one or more carrier materials and/or one or more liquid solvents.

Further processing steps may precede and/or follow, such as drying (e.g. freeze-drying (lyophilization), spray-drying and evaporation), granulation, agglomeration, concentrating (e.g. to syrups, formed via concentration and/or with the aid of thickeners), pasteurizing, sterilizing, freezing, dissolving, dispersing, filtering, centrifuging, confectioning, and the like.

When an extract formulation according to the present invention or an extract formulation obtained according to a method of the present is added to a food product or pharmaceutical or nutraceutical, this also results in a functional food product or pharmaceutical or nutraceutical composition according to the invention.

Further additives may be included, such as vitamins, minerals, e.g. in the form of mineral salts, unsaturated fatty acids or oils or fats comprising them, other extracts, or the like.

The functional food products according to the invention may be of any food type. They may comprise one or more common food ingredients in addition to the food product, such as flavours, fragrances, sugars, minerals, vitamins, stabilizers, thickeners, dietary fibers, protein, amino acids or the like in appropriate amounts, or mixtures of two or more thereof, in accordance with the desired type of food product.

Examples of basic food products and thus of functional food products according to the invention are fruit or juice products, such as orange and grapefruit, tropical fruits, banana, apple, peach, blackberry, cranberry, plum, prune, apricot, cherry, peer, strawberry, marionberry, black currant, red currant, tomato, vegetable, e.g. carrot, or blueberry juice, soy-based beverages, or concentrates thereof, respectively; lemonades; extracts, e.g. coffee, tea, green tea; dairy type products, such as milk, dairy spreads, quark, cheese, cream cheese, custards, puddings, mousses, milk type drinks and yoghurt; frozen confectionery products, such as ice-cream, frozen yoghurt, sorbet, ice milk, frozen custard, water-ices, granitas and frozen fruit purees; baked goods, such as bread, cakes, biscuits, cookies or crackers; spreads, e.g. margarine, butter, peanut butter honey; snacks, e.g. chocolate bars, muesli bars; pasta products or other cereal products, such as muesli; ready-to-serve-dishes; frozen food; tinned food; syrups; oils, such as salad oil; sauces, such as salad dressings, mayonnaise; fillings; dips; chewing gums; sherbet; spices; cooking salt; instant drink powders, such as instant coffee, instant tee or instant cocoa powder; instant powders e.g. for pudding or other desserts; meat fish or fish or meat products, such as sausages, burgers, meat loafs, meatballs, meat extracts, canned or tinned fish or meat, meat vol-au-vent, meat or fish soup, meat or fish skewers, fish fingers; or the like.

One or more other customary additives may be present, such as flavour, fragrances or other additives, such as one or more selected from stabilizers, e.g. thickeners; coloring agents, such as edible pigments or food dyes; bulking agents, polyols, such as xylitol, mannitol, maltitol or the like; preservatives, such as sodium or potassium benzoate, sodium or calcium carbonate or other food grade preservatives; antioxidants, such as ascorbic acid, carotinoids, tocopherols or polyphenols; mono-, oligo- or polysaccharides, such as glucose, fructose, sucrose, soy-oligosaccharides, xylo-oligosaccharides, galacto-oligosaccharides; other artificial or natural non- or low-caloric sweeteners, such as aspartame or acesulfame; bitterness blockers; acidifiers in the form of edible acids, such as citric acids, acetic acid, lactic acid, adipic acid; flavours, e.g. artificial or natural (e.g. botanical flavours); emulsifiers; diluents; wetting agents, e.g. glycerol; stabilizers; coatings; isotonic agents; absorption promoting or delaying agents; and the like.

An extract formulation according to the invention or produced according to a method of the present invention can also be included in confectioned preparations to be added to foods including beverages, e.g. in the form of powders or granules, e.g. freeze-dried or spray-dried, concentrates, solutions, dispersions or other instant form, or the like.

In another aspect, the present invention relates to an extract formulation obtained according to a method according to the present invention, preferably in one of the preferred embodiments indicated above, or an extract formulation according to the present invention, preferably in one of the preferred embodiments indicated above, for use in a therapeutic or prophylactic method
for treating a disease attendant on hyperglycemia or of a carbohydrate metabolic disorder, preferably selected from the group consisting of prediabetes, obesity, hyperlipemia, in particular carbohydrate-induced hyperlipemia (i.e. elevated blood lipids, particularly triglycerides, after carbohydrate ingestion; sometimes used synonymously with hyperlipoproteinemia type IV or V phenotypes), or the genetic disorders causing them), arteriosclerosis, arteriolosclerosis, atherosclerosis, and/or diabetes mellitus, in particular type 2 diabetes (type II diabetes, sometimes still called non insulin dependent diabetes mellitus),

and/or
for treating metabolic syndrome (also called metabolic syndrome X or syndrome X),

and/or
for reducing the degradation of ingested carbohydrates, particularly of one or more polysaccharides, preferably polysaccharides comprising ten or more glucose units, particularly preferably comprising ten or more α-D-glucose units, especially comprising amylose and/or amylopectin,

and/or
for controlling, preferably lowering, glycemia (i.e. the blood sugar level and/or preventing a high blood sugar level, in particular lowering postprandial blood glucose concentration), preferably in a mammal, especially in a human being,

and/or
for treating or preventing postprandial hyperglycemia.

In another aspect, the present invention relates to the use of an extract obtained by extraction of plant material of Rhodamnia cinerea, said plant material preferably comprising, essentially consisting or consisting of leaves and/or roots of Rhodamnia cinerea, with an extractant essentially consisting or consisting of water or a mixture essentially consisting or consisting of an alcohol having 1 to 3 carbon atoms and water, preferably of an extract formulation produced by a method according to the present invention, preferably in one of the preferred embodiments indicated above, or an extract formulation according to the present invention, preferably in one of the preferred embodiments indicated above,
as alpha-amylase inhibitor, preferably as pancreatic alpha-amylase and/or salivary alpha-amylase (ptyalin) inhibitor,

and/or
for reducing the activity of mammalian, preferably human alpha-amylase,

and/or
in a food composition, a nutraceutical composition or a food supplement,

and/or
for the manufacture of a food composition, a nutraceutical composition or a food supplement.

In yet another aspect, the present invention relates to a composition, preferably an orally administrable composition, comprising

an effective amount of an extract formulation as obtained by a method according to the present invention or an extract formulation according to the present invention, said effective amount being sufficient
to reduce alpha-amylase activity in vitro, preferably to reduce (preferably porcine pancreatic) alpha-amylase activity in vitro by 10% or more, preferably by 20% or more, more preferably by 30% or more, most preferably by 50% or more,

and/or
to reduce the glycemic response to orally administered wheat starch, preferably by oral gavage of a 7.5 wt. % solution of wheat starch in water, in an amount of 1.5 g/kg in vivo in rats, preferably in normal male Wistar rats, by 10% or more, preferably by 15% or more, more preferably by 20% or more, measured 30 minutes after oral administration of the wheat starch.

Preferably, the amount of an extract formulation according to the present invention is in the range of 0.5 to 99 wt. %, preferably of 1 to 75 wt. %, more preferably of 2 to 60 wt. %, based on the total weight of the composition. Particularly preferably, the amount of an extract formulation according to the present invention is 5 wt. % or greater, especially preferably 10 wt. % or greater, most preferably 15 wt. % or greater, and most preferably 20 wt. % or greater, in each case based on the total weight of the composition.

Optionally, a composition according to the present invention may additionally comprise one or more further liquid or solid carrier substances.

Since extract formulations according to the present invention or produced according to a method of the present invention inhibit alpha-amylase—i.e. one of the different important enzymes involved in the digestion of ingested carbohydrates and thereby influencing postprandial blood glucose concentration (as already explained above)—in many cases it is advantageous and preferred to combine an extract formulation according to the present invention or produced according to a method of the present invention with other enzyme inhibitors, e.g. like further glycosidase inhibitors, further antidiabetic active substances, and/or glycogen phosphorylase inhibitors.

Therefore, a preferred composition according to the present invention additionally comprises one or more further glycosidase (also called glycoside hydrolase; enzyme classification EC 3.2.1) inhibitors, wherein said glycosidase inhibitors are preferably selected from component (b) consisting of

oligo-1,6-glucosidase (also called isomaltase; enzyme classification EC 3.2.1.10; systematic name: oligosaccharide α-1,6-glucohydrolase) inhibitors, alpha-glucosidase (also called maltase or maltase-glucoamylase; systematic name: α-D-glucoside glucohydrolase; enzyme classification EC 3.2.1.20) inhibitors, amylo-alpha-1,6-glucosidase (systematic name: glycogen phosphorylase-limit dextrin 6-α-glucohydrolase; enzyme classification EC 3.2.1.33) inhibitors, sucrose alpha-glucosidase (also called sucrase, sucrase-isomaltase; enzyme classification EC 3.2.1.48) inhibitors, isoamylase (2R,3R,4R,5S)-1-(2-hydroxyethyl)-2-(hydroxymethyl)-3,4,5-piperidinetriol, and the various 3,4,5-trihydroxypiperidines related thereto, are disclosed in U.S. Pat. No. 4,639,436. The glucosidase inhibitor emiglitate, ethyl p-[2-[(2R,3R,4R,5S)-3,4,5-trihydroxy-2-(hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor MDL-25637, 2,6-dideoxy-7-O-beta-D-glucopyrano-syl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor camiglibose, methyl 6-deoxy-6-[(2R,3R,4R,5S)-3,4,5-trihydroxy-2-(hydroxymethyl)piperidino]-alpha-D-glucopyranoside sesquihydrate, the deoxy-nojirimycin derivatives related thereto, the various pharmaceutically acceptable salts thereof and synthetic methods for the preparation thereof, are disclosed in U.S. Pat. No. 5,157,116 and U.S. Pat. No. 5,504,078. The glucosidase inhibitor pradimicin-Q and a process for the preparation thereof by the microbial cultivation of Actinomadura verrucospora strains as disclosed in U.S. Pat. No. 5,091,418 and U.S. Pat. No. 5,217,877. The glycosidase inhibitor salbostatin, the various pseudosaccharides related thereto, the various pharmaceutically acceptable salts thereof and a process for the preparation thereof by the microbial cultivation of a Streptomyces albus strain as disclosed in U.S. Pat. No. 5,091,524.

A variety of other amylase inhibitors are known to one of ordinary skill in the art. However, in the practice of the methods and compositions of the instant invention, certain amylase inhibitors are preferred.

The amylase inhibitor tendamistat, the various cyclic peptides related thereto and processes for the preparation thereof by the microbial cultivation of certain Streptomyces strains as disclosed in U.S. Pat. No. 4,451,455. The amylase inhibitor AI-3688, the various cyclic polypeptides related thereto, and a process for the preparation thereof by the microbial cultivation of a Streptomyces aureofaciens strain as disclosed in U.S. Pat. No. 4,623,714. The amylase inhibitor trestatin, preferably consisting of a mixture of trestatin A, trestatin B and trestatin C, the various trehalose-containing aminosugars related thereto and a process for the preparation thereof by the microbial cultivation of certain Streptomyces strains as disclosed in U.S. Pat. No. 4,273,765.

In a preferred aspect, the present invention relates to compositions having (further) improved properties, in particular exhibiting improved efficacy and/or an improved time activity profile, comprising one or more further glycosidase inhibitors, wherein preferably one, a plurality or all of the further glycosidase inhibitors are selected from the group consisting of

acarbose, miglitol, voglibose, camiglibose, pradimicin-Q, saponarin, mahanimbine, gymnemic acids, S-allyl cysteine sulphoxide, nojirimycin, 1-deoxynojirimycin (moranoline), N-methyl-1-deoxynojirimycin, deoxygalactonojirimycin, emiglitate, adiposines (preferably adiposine 1, adiposine 2), swainsonine, australine (1R,2R,3R,7S,7aR)-3-hydroxymethyl-1,2,7-trihydroxypyrrolizidine), 2-amino-3,4-dihydroxy-5-methoxybenzoic acid, castanospermine, 6-epicastanospermine, 2,5-dihydroxymethyl-3,4-dihydroxypyrrolidine (DMDP), salacinol, kotalanol, fustin, fisetin, gallic acid, methyl gallate, 3′,4′,7′-trihydroxyflavone, (−)-3-O-galloylepicatechin, (−)-3-O-galloylcatechin, epicatechin, salbostatin, and the pharmaceutically acceptable salts thereof,

extracts, dried extracts or dried parts of vegetal organisms selected from the group consisting of (preferably leaves and/or fruits of) Aegle marmeloes, (preferably fleshy leaves of) Aloe vera, (preferably root and/or root bark of) Anacardium occidentale, (preferably aerial parts of) Artemisia santolina, (preferably tubers and/or fleshy roots of) Asparagas racemosus, (preferably roots and/or aerial parts of) Berberis integrimma, (preferably seeds of) Brassica nigra, (preferably leaves of) Camellia sinensis, (preferably seeds of) Cannabis sativa, (preferably leaves, bark, flowers and/or seeds of) Cassia auriculata, (preferably flowers of) Cassia fistula, (preferably roots of) Cichorium intybus, (preferably flowers of) Citrus aurantium, (preferably tubers of) Coccinia indica, (preferably leaves of) Crocus sativa, (preferably seeds of) Cuminum cymirum, (preferably seeds, roots and/or fruits of) Eugenia jambolana, (preferably bark, leaves, fruits and/or flowers of) Ficus bengalensis, (preferably leaves of) Ficus carica, (preferably fruits of) Foeniculum vulgare, (preferably aerial parts of) Glycyrrhiza glabra, (preferably leaves, flowers, bark and/or fruits of) Gossypium arboreturn, (preferably bark and/or seeds of) Holarina antidysentrica, (preferably leaves of) Lawsonia inermis, (preferably seeds of) Nigella sativa, Phyllanthus amarus, (preferably fruits of) Piper nigrum, (preferably fruits or fruits hulls of) Punica granatum, (preferably fruits of) Solanum dulcamara, (preferably seeds of) Strychnos potatorum, (preferably bark of) Terminalia arjuna, (preferably fruits of) Terminalia chebulla, (preferably fruit bodies of) maitake mushroom (Grifola frondosa), (preferably fruits of) Schizandra chinensis, (preferably leaves of) Gymnea sylvestre, (preferably fruits of) bitter melon (Momordica charantia), (preferably seeds of) fenugreek (Trigonella foenum graecum), (preferably bark of) Pterocarpus marsupium, (preferably leaves of) Murraya koenigii, (preferably leaves of) Ocimum sanctum, (preferably bark or leaves of) Tinospora cordifolia, (preferably seed kernels of) Syzygium cumini, (preferably rhizome of) ginger (Zingiber officinale), (preferably bulbs or cloves of) garlic (Allium sativum), (preferably roots and/or stem of) plants of the genus Salacia, (preferably seeds of) plants of the genus Oenothera, (preferably leaves of) plants of the genus Morus (mulberry, preferably Morus alba, Morus australis, Morus rubra, and Morus nigra), Phyllantus nirunri, Smilax officinalis, Yerba Mate (Ilex paraguayensis), Tagetes minuta, (preferably fruits, leaves, roots and/or stem of) Solanum diphyllum, (preferably stem of) Rhus verniciflua, Rumex nepalensis, Olea europaea, (preferably leaves of) Malpighia glabra, Cornus officinalis, Pelvetia wrightii, Syzygium aromaticum, (preferably seeds or seed coat of) Tamarindus indica, Camellia ptilophylla, Hydrangea paniculata, Rubus phoenicolasius, Chrysanthemum coronarium, (preferably leaves of) Cyclocarya paliurus, Cymbopogon martinii, (preferably pericarp and/or bark of) Castanea crenata,

L-arabinose, L-fucose, D-xylose, L-xylose, D-ribose, D-tagatose, D-ribulose, D-lyxose, D-xylulose,

extracts, preferably dried extracts, of Alstonia scholaris, Piper umbellatum, Tussilago farfara, Terminalia chebula, Bergenia cilata, Grateloupia elliptica, Syagrus romanzoffiana, Fagara tessmannii, Gypsophila oldhamiana,

vasicine, vasicinol, piperumbellactams (preferably piperumbellactam A, piperumbellactam B and piperumbellactam C), chebulanin, chebulagic acid, chebulinic acid, β-hydroxykompasinol A, kompasinol A, scirpusin A, scirpusin C, pentahydroxystilbene, curcumin, demethoxycurcumin, bisdemethoxycurcumin, 3b-acetoxy-16b-hydroxybetulinic acid, cyanidin-3-galactoside,

extracts, preferably dried extracts, of Lagerstoemia speciosa, Camellia sinensis, guava leaves (Psidium guajava), Anacardium occidentale, Syzygium zeylanicum, Cleistocalyx operculatus, Horsefieldia amygdalina, Careya arborea, Phyllanthus amarus, Acanthopanax sieboldianum, (preferably bark of) Ficus bengalensis, (preferably seeds of) Syzygium cumini, (preferably leaves of) Cinnamonum verum, (preferably rhizome of) Curcuma longa, (preferably leaves of) Bixa orellana, (preferably leaves of) Murraya koenigii, (preferably seeds of) Tribulus terrestris, (preferably fruits, leaves, roots and/or stem of) Solanum diphyllum, (preferably seeds and/or seed shells of) Japanese horse chestnut (Aesculus turbinate), (preferably stem and/or bark of) Callistemon rigidus, (preferably roots, bark and/or stem of) plants of the genus Morus, kidney beans (Phaseolus sp.), white kidney beans (Phaseolus vulgaris), (preferably seeds of) wheat (Triticum spp.), (preferably leaves of) Pistacia atlantica, (preferably aerial parts of) Sarcopoterium spinosum, (preferably roots and/or rhizome of) Rheum ribes,

dicaffeoylquinic acids (preferably 3,4-dicaffeoylquinic acid, 3,5-dicaffeoylquinic acid, 4,5-dicaffeoylquinic acid), oleanolic acid, ursolic acid, lupeol, phaseolamin, scirpusin B, piceatannol, trestatins (preferably trestatin A, trestatin B and trestatin C), tendamistat, and AI-3688.

Preferred compositions according to the present invention comprise an extract formulation obtained according to a method according to the present invention, preferably in one of the preferred embodiments indicated above, or an extract formulation according to the present invention, preferably in one of the preferred embodiments indicated above, and one or more alpha-glucosidase inhibitors, wherein preferably one, a plurality or all of the alpha-glucosidase inhibitors of component (b) are selected from the group consisting of

(b-i) acarbose, miglitol, voglibose, camiglibose, pradimicin-Q, saponarin, mahanimbine, gymnemic acids, S-allyl cysteine sulphoxide, nojirimycin, 1-deoxynojirimycin (moranoline), N-methyl-1-deoxynojirimycin, deoxygalactonojirimycin, emiglitate, adiposines (preferably adiposine 1, adiposine 2), swainsonine, australine (1R,2R,3R,7S,7aR)-3-hydroxymethyl-1,2,7-trihydroxypyrrolizidine), 2-amino-3,4-dihydroxy-5-methoxybenzoic acid, castanospermine, 6-epicastanospermine, 2,5-dihydroxymethyl-3,4-dihydroxypyrrolidine (DMDP), salacinol, kotalanol, fustin, fisetin, gallic acid, methyl gallate, 3′,4′,7′-trihydroxyflavone, (−)-3-O-galloylepicatechin, (−)-3-O-galloylcatechin, epicatechin, salbostatin, or pharmaceutically acceptable salts thereof,

(b-ii) extracts, dried extracts or dried parts of vegetal organisms selected from the group consisting of (preferably leaves and/or fruits of) Aegle marmeloes, (preferably fleshy leaves of) Aloe vera, (preferably root and/or root bark of) Anacardium occidentale, (preferably aerial parts of) Artemisia santolina, (preferably tubers and/or fleshy roots of) Asparagas racemosus, (preferably roots and/or aerial parts of) Berberis integrimma, (preferably seeds of) Brassica nigra, (preferably leaves of) Camellia sinensis, (preferably seeds of) Cannabis sativa, (preferably leaves, bark, flowers and/or seeds of) Cassia auriculata, (preferably flowers of) Cassia fistula, (preferably roots of) Cichorium intybus, (preferably flowers of) Citrus aurantium, (preferably tubers of) Coccinia indica, (preferably leaves of) Crocus sativa, (preferably seeds of) Cuminum cymirum, (preferably seeds, roots and/or fruits of) Eugenia jambolana, (preferably bark, leaves, fruits and/or flowers of) Ficus bengalensis, (preferably leaves of) Ficus carica, (preferably fruits of) Foeniculum vulgare, (preferably aerial parts of) Glycyrrhiza glabra, (preferably leaves, flowers, bark and/or fruits of) Gossypium arboreturn, (preferably bark and/or seeds of) Holarina antidysentrica, (preferably leaves of) Lawsonia inermis, (preferably seeds of) Nigella sativa, Phyllanthus amarus, (preferably fruits of) Piper nigrum, (preferably fruits or fruits hulls of) Punica granatum, (preferably fruits of) Solanum dulcamara, (preferably seeds of) Strychnos potatorum, (preferably bark of) Terminalia arjuna, (preferably fruits of) Terminalia chebulla, (preferably fruit bodies of) maitake mushroom (Grifola frondosa), (preferably fruits of) Schizandra chinensis, (preferably leaves of) Gymnea sylvestre, (preferably fruits of) bitter melon (Momordica charantia), (preferably seeds of) fenugreek (Trigonella foenum graecum), (preferably bark of) Pterocarpus marsupium, (preferably leaves of) Murraya koenigii, (preferably leaves of) Ocimum sanctum, (preferably bark or leaves of) Tinospora cordifolia, (preferably seed kernels of) Syzygium cumini, (preferably rhizome of) ginger (Zingiber officinale), (preferably bulbs or cloves of) garlic (Allium sativum), (preferably roots and/or stem of) plants of the genus Salacia, (preferably seeds of) plants of the genus Oenothera, (preferably leaves of) plants of the genus Morus (mulberry, preferably Morus alba, Morus australis, Morus rubra, and Morus nigra), Phyllantus niruri, Smilax officinalis, Yerba Mate (Ilex paraguayensis), Tagetes minuta, (preferably fruits, leaves, roots and/or stem of) Solanum diphyllum, (preferably stem of) Rhus verniciflua, Rumex nepalensis, Olea europaea, (preferably leaves of) Malpighia glabra, Cornus officinalis, Pelvetia wrightii, Syzygium aromaticum, (preferably seeds or seed coat of) Tamarindus indica, Camellia ptilophylla, Hydrangea paniculata, Rubus phoenicolasius, Chrysanthemum coronarium, (preferably leaves of) Cyclocarya paliurus, Cymbopogon martinii, and (preferably pericarp and/or bark of) Castanea crenata.

Further preferred compositions according to the present invention comprise one or more alpha-glucosidase inhibitors, wherein one, a plurality or all of the alpha-glucosidase inhibitors of component (b) are selected from the group consisting of

(b-i) acarbose, miglitol, voglibose, saponarin, mahanimbine, swainsonine, castanospermine, 6-epicastanospermine, salacinol, kotalanol, gallic acid, and (−) epicatechin,

and

(b-ii) extracts, preferably in dried form, preferably aqueous, alcoholic or aqueous alcoholic extracts in dried form, of vegetal organisms selected from the group consisting of (preferably fruit bodies of) maitake mushroom (Grifola frondosa), (preferably fruits of) Schizandra chinensis, (preferably leaves of) Gymnea sylvestre, (preferably fruits of) bitter melon (Momordica charantia), (preferably seeds of) fenugreek (Trigonella foenum graecum), (preferably bark of) Pterocarpus marsupium, (preferably leaves of) Murraya koenigii, (preferably leaves of) Ocimum sanctum, (preferably leaves of) Tinospora cordifolia, (preferably seed kernels of) Syzygium cumini, (preferably rhizome of) ginger (Zingiber officinale), (preferably bulbs or cloves of) garlic (Allium sativum), (preferably roots and/or stem of) Salacia reticulata and (preferably roots and/or stem of) Salacia oblonga.

Preferably, one, a plurality or all of the further alpha-amylase inhibitors of component (b) are selected from the group consisting of extracts, preferably dried extracts, of Lagerstoemia speciosa, Camellia sinensis, guava leaves (Psidium guajava), Anacardium occidentale, Syzygium zeylanicum, Cleistocalyx operculatus, Horsefieldia amygdalina, Careya arborea, Phyllanthus amarus, Acanthopanax sieboldianum, (preferably bark of) Ficus bengalensis, (preferably seeds of) Syzygium cumini, (preferably leaves of) Cinnamonum verum, (preferably rhizome of) Curcuma longa, (preferably leaves of) Bixa orellana, (preferably leaves of) Murraya koenigii, (preferably seeds of) Tribulus terrestris, (preferably fruits, leaves, roots and/or stem of) Solanum diphyllum, (preferably seeds and/or seed shells of) Japanese horse chestnut (Aesculus turbinate), (preferably stem and/or bark of) Callistemon rigidus, (preferably roots, bark and/or stem of) plants of the genus Morus, kidney beans (Phaseolus sp.), white kidney beans (Phaseolus vulgaris), (preferably seeds of) wheat,

dicaffeoylquinic acids (preferably 3,4-dicaffeoylquinic acid, 3,5-dicaffeoylquinic acid, 4,5-dicaffeoylquinic acid), oleanolic acid, ursolic acid, lupeol, phaseolamin, scirpusin B, piceatannol, trestatins (preferably trestatin A, trestatin B and trestatin C), tendamistat, and AI-3688.

In another aspect, the present invention relates to a composition according to the present invention (as defined above), additionally comprising one or more glycogen phosphorylase inhibitors and/or one or more further antidiabetic active compounds.

Such compositions have (further) improved properties, and in particular exhibit broader efficacy, improved efficiency, and/or show an improved time activity profile, resulting in an even better overall performance.

A review of antidiabetic plants traditionally used in South African herbal medicine for treatment of diabetes is given in J. Clin. Biochem. Nutr. 2010, 47, 98-106. Alternatively or additionally, thiazolidinediones (also known as glitazones) may also be used as further antidiabetic actives in combination with an extract formulation according to the present invention.

Examples of glycogen phosphorylase inhibitors that can be used according to the present invention in combination with an extract formulation of the present invention include those mentioned in US 2001/0046956 A1, in particular: 6H-thieno[2,3-b]pyrrole-5-carboxylic acid [(1S)-benzyl-3-((3R,4S)-dihydroxy-pyrrolidin-1-yl)-(2R)-hydroxy-3-oxo-propyl]-amide; 2-bromo-6H-thieno[2,3-b]pyrrole-5-carboxylic acid [(1S)-benzyl-3-((3R,4S)-dihydroxy-pyrrolidin-1yl)-(2R)-hydroxy-3-oxo-propyl]-amide; 2-methyl-6H-thieno[2,3-b]pyrrole-5-carboxylic acid [(1S)-benzyl-3-((3R,4S)-dihydroxy-pyrrolidin-1-yl)-(2R)-hydroxy-3-oxo-propyl]-amide; (+−)-2-methyl-6H-thieno[2,3-b]pyrrole-5-carboxylic acid [1-benzyl-2-((3R,4S)-dihydroxy-pyrrolidin-1yl)-2-oxo-ethyl]-amide; 2-bromo-6H-thieno[2,3-b]pyrrole-5-carboxylic acid [(1S)-benzyl-2-((3R,4S)-dihydroxy-pyrrolidin-1yl)-2-oxo-ethyl]-amide; 2-chloro-6H-thieno[2,3-b]pyrrole-5-carboxylic acid [(1S)-benzyl-3-((3R,4S)-dihydroxy-pyrrolidin-1-yl)-(2R)-hydroxy-3-oxo-propyl]-amide; 2-chloro-6H-thieno[2,3-b]pyrrole-5-carboxylic acid [(1S)-benzyl-2-((3R,4S)-dihydroxy-pyrrolidin-1yl)-2-oxo-ethyl]-amide; 2,4-dichloro-6H-thieno[2,3-b]pyrrole-5-carboxylic acid [(1S)-benzyl-3-((3R,4S)-dihydroxy-pyrrolidin-1yl)-(2R)-hydroxy-3-oxo-propyl]-amide; (+−)-4H-thieno[3,2-b]pyrrole-5-carboxylic acid [1-benzyl-2-((3R,4S)-dihydroxy-pyrrolidin-1yl)-2-oxo-ethyl]-amide; 2-bromo-4H-thieno[3,2-b]pyrrole-5-carboxylic acid [(1S)-benzyl-3-((3R,4S)-dihydroxy-pyrrolidin-1yl)-(2R)-hydroxy-3-oxo-propyl]-amide; 4H-thieno[3,2-b]pyrrole-5-carboxylic acid [(1S)-benzyl-3-((3R,4S)-dihydroxy-pyrrolidin-1yl)-(2R)-hydroxy-3-oxo-propyl]-amide; (+−)-2-bromo-4H-furo[3,2-b]pyrrole-5-carboxylic acid [1-benzyl-2-((3R,4S)-dihydroxy-pyrrolidin-1yl)-2-oxo-ethyl]-amide; 2-bromo-4H-furo[3,2-b]pyrrole-5-carboxylic acid [(1S)-benzyl-3-((3R,4S)-dihydroxy-pyrrolidin-1yl)-(2R)-hydroxy-3-oxo-propyl]-amide; 6H-thieno[2,3-b]pyrrole-5-carboxylic acid [(1S)-benzyl-2-((3R,4S)-dihydroxy-pyrrolidin-1yl)-2-oxo-ethyl]-amide; 2-bromo-4H-thieno[3,2-b]pyrrole-5-carboxylic acid [(1S)-benzyl-2-((3R,4S)-dihydroxy-pyrrolidin-1yl)-2-oxo-ethyl]-amide; 2-methyl-4H-thieno[3,2-b]pyrrole-5-carboxylic acid [(1S)-benzyl-2-((3R,4S)-dihydroxy-pyrrolidin-1yl)-2-oxo-ethyl]-amide; 2,4-dichloro-6H-thieno[2,3-b]pyrrole-5-carboxylic acid [(1S)-benzyl-2-((3R,4S)-dihydroxy-pyrrolidin-1yl)-2-oxo-ethyl]-amide; 2-cyano-6H-thieno[2,3-b]pyrrole-5-carboxylic acid [(1S)-benzyl-2-(3-hydroxy-azetidin-1yl)-2-oxo-ethyl]-amide; 2-chloro-6H-thieno[2,3-b]pyrrole-5-carboxylic acid [(1S)-benzyl-2-morpholin-4-yl-2-oxo-ethyl]-amide; 2-chloro-6H-thieno[2,3-b]pyrrole-5-carboxylic acid [(1S)-dimethylcarbamoyl-2-phenyl-ethyl]-amide; 2-chloro-6H-thieno[2,3-b]pyrrole-5-carboxylic acid [(1S)-benzyl-2-(1,1-dioxo-1-thiazolidin-3-yl)-2-oxo-ethyl]-amide; 1-{(2S)-[(2-chloro-6H-thieno[2,3-b]pyrrole-5-carbonyl)-amino]-3-phenyl-propionyl}-piperidine-4-carboxylic acid ethyl ester; 2-bromo-6H-thieno[2,3-b]pyrrole-5-carboxylic acid [(1S)-benzyl-2-(3-hydroxy-azetidin-1yl)-2-oxo-ethyl]-amide; 2-methyl-4H-furo[3,2-b]pyrrole-5-carboxylic acid [(1S)-benzyl-2-((3R,4S)-dihydroxy-pyrrolidin-1yl)-2-oxo-ethyl]-amide; 2-trimethylsilanylethynyl-6H-thieno[2,3-b]pyrrole-5-carboxylic acid [(1S)-benzyl-2-(3-hydroxy-azetidin-1yl)-2-oxo-ethyl]-amide; 2-ethynyl-6H-thieno[2,3-b]pyrrole-5-carboxylic acid [(1S)-benzyl-2-(3-hydroxy-azetidin-1-yl)-2-oxo-ethyl]-amide; 2-fluoro-4H-thieno[3,2-b]pyrrole-5-carboxylic acid [(1S)-benzyl-2-((3R,4S)-dihydroxy-pyrrolidin-1-yl)-2-oxo-ethyl]-amide; 2-cyano-4H-furo[3,2-b]pyrrole-5-carboxylic acid [(1S)-benzyl-2-(3-hydroxy-azetidin-1yl)-2-oxo-ethyl]-amide; 2-chloro-4H-furo[3,2-b]pyrrole-5-carboxylic acid [(1S)-benzyl-2-((3R,4S)-dihydroxy-pyrrolidin-1yl)-2-oxo-ethyl]-amide; 2-chloro-4H-furo[3,2-b]pyrrole-5-carboxylic acid [(1S)-benzyl-3-((3R,4S)-dihydroxy-pyrrolidin-1-yl)-(2R)-hydroxy-3-oxo-propyl]-amide; 1-{(2S)-[(2-chloro-6H-thieno[2,3-b]pyrrole-5-carbonyl)-amino]-3-phenyl-propionyl}-piperidine-4-carboxylic acid; 3-chloro-4H-thieno[3,2-b]pyrrole-5-carboxylic acid [(1S)-benzyl-2-((3R,4S)-dihydroxy-pyrrolidin-1yl)-2-oxo-ethyl]-amide; 3-chloro-4H-thieno[3,2-b]pyrrole-5-carboxylic acid [(1S)-benzyl-3-((3R,4S)-dihydroxy-pyrrolidin-1yl)-(2R)-hydroxy-3-oxo-propyl]-amide; 3-bromo-4H-thieno[3,2-b]pyrrole-5-carboxylic acid [(1S)-benzyl-2-((3R,4S)-dihydroxy-pyrrolidin-1yl)-2-oxo-ethyl]-amide; 3-bromo-4H-thieno[3,2-b]pyrrole-5-carboxylic acid [(1S)-benzyl-3-((3R,4S)-dihydroxy-pyrrolidin-1yl)-(2R)-hydroxy-3-oxo-propyl]-amide; 2-chloro-4H-thieno[3,2-b]pyrrole-5-carboxylic acid [(1S)-benzyl-3-((3R,4S)-dihydroxy-pyrrolidin-1yl)-(2R)-hydroxy-3-oxo-propyl]-amide; 2-chloro-4H-thieno[3,2-b]pyrrole-5-carboxylic acid [(1S)-benzyl-2-((3R,4S)-dihydroxy-pyrrolidin-1yl)-2-oxo-ethyl]-amide; 3-methyl-4H-thieno[3,2-b]pyrrole-5-carboxylic acid [(1S)-benzyl-2-((3R,4S)-dihydroxy-pyrrolidin-1yl)-2-oxo-ethyl]-amide; 3-methyl-4H-thieno[3,2-b]pyrrole-5-carboxylic acid [(1S)-benzyl-3-((3R,4S)-dihydroxy-pyrrolidin-1yl)-(2R)-hydroxy-3-oxo-propyl]-amide; 2-cyano-4H-thieno[3,2-b]pyrrole-5-carboxylic acid [(1S)-benzyl-2-((3R,4S)-dihydroxy-pyrrolidin-1yl)-2-oxo-ethyl]-amide; 2-cyano-4H-furo[3,2-b]pyrrole-5-carboxylic acid [(1S)-benzyl-3-((3R,4S)-dihydroxy-pyrrolidin-1yl)-(2R)-hydroxy-3-oxo-propyl]-amide; 3-bromo-4H-furo[3,2-b]pyrrole-5-carboxylic acid [(1S)-benzyl-2-((3R,4S)-dihydroxy-pyrrolidin-1yl)-2-oxo-ethyl]-amide; 3-bromo-4H-furo[3,2-b]pyrrole-5-carboxylic acid [(1S)-benzyl-3-((3R,4S)-dihydroxy-pyrrolidin-1yl)-(2R)-hydroxy-3-oxo-propyl]-amide; 4H-1,7-dithia-4-aza-cyclopenta[a]pentalene-5-carboxylic acid [(1S)-benzyl-3-((3R,4S)-dihydroxy-pyrrolidin-1yl)-(2R)-hydroxy-3-oxo-propyl]-amide; 4H-1,7-dithia-4-aza-cyclopenta[a]pentalene-5-carboxylic acid [(1S)-benzyl-2-((3R,4S)-dihydroxy-pyrrolidin-1yl)-2-oxo-ethyl]-amide; 2-chloro-3-methyl-4H-thieno[3,2-b]pyrrole-5-carboxylic acid [(1S)-benzyl-2-((3R,4S)-dihydroxy-pyrrolidin-1yl)-2-oxo-ethyl]-amide; 2-chloro-3-methyl-4H-thieno[3,2-b]pyrrole-5-carboxylic acid [(1S)-benzyl-3-((3R,4S)-dihydroxy-pyrrolidin-1yl)-(2R)-hydroxy-3-oxo-propyl]-amide; 2-methylsulfanyl-4H-thieno[3,2-b]pyrrole-5-carboxylic acid [(1S)-benzyl-2-((3R,4S)-dihydroxy-pyrrolidin-1yl)-2-oxo-ethyl]-amide; 2-bromo-4H-thieno[3,2-b]pyrrole-5-carboxylic acid [(1S)-benzyl-2-(3-hydroxy-azetidin-1yl)-2-oxo-ethyl]-amide; 2-bromo-4H-thieno[3,2-b]pyrrole-5-carboxylic acid [(1S)-benzyl-2-(1,1-dioxo-1-thiazolidin-3-yl)-2-oxo-ethyl]-amide; 2-bromo-4H-thieno[3,2-b]pyrrole-5-carboxylic acid [(1S)-benzyl-2-morpholin-4-yl-2-oxo-ethyl]-amide; 2-bromo-4H-thieno[3,2-b]pyrrole-5-carboxylic acid [(1S)-benzyl-2-((3S,4S)-dihydroxy-pyrrolidin-1-yl)-2-oxo-ethyl]-amide; 2-bromo-4H-thieno[3,2-b]pyrrole-5-carboxylic acid [(1S)-benzyl-2-((3R,4R)-dihydroxy-pyrrolidin-1yl)-2-oxo-ethyl]-amide; 2-bromo-4H-thieno[3,2-b]pyrrole-5-carboxylic acid [(1S)-benzyl-2-(4-hydroxy-piperidin-1-yl)-2-oxo-ethyl]-amide; and the pharmaceutically acceptable salts thereof.

Methods for manufacturing the glycogen phosphorylase inhibitors listed above can be found in U.S. Pat. No. 6,828,343.

WO 96/39384 and WO 96/39385 disclose additional glycogen phosphorylase inhibitors that can be used in combination with an extract formulation according to the present invention. Additional preferred glycogen phosphorylase inhibitors include:

5-chloro-1H-indole-2-carboxylic acid [(1S)—((R)-hydroxy-dimethylcarbamoyl-methyl)-2-phenyl-ethyl]-amide; 5,6-dichloro-1H-indole-2-carboxylic acid {(1S)-[(R)-hydroxy-(methoxy-methyl-carbamoyl)-methyl]-2-phenyl-ethyl}-amide; 5-chloro-1H-indole-2-carboxylic acid {(1S)-[(R)-hydroxy-(methoxy-methyl-carbamoyl)-methyl]-2-phenyl-ethyl}-amide; 5-chloro-1H-indole-2-carboxylic acid ((1S)-{(R)-hydroxy-[(2-hydroxy-ethyl)-methyl-carbamoyl]-methyl}-2-phenyl-ethyl)-amide; 5-chloro-1H-indole-2-carboxylic acid [(1S)-benzyl-3-((3R,4S)-dihydroxy-pyrrolidin-1yl)-(2R)-hydroxy-3-oxo-propyl]-amide; 5-chloro-1H-indole-2-carboxylic acid {(1S)-[(R)-hydroxy-(methyl-pyridin-2-yl-carbamoyl)-methyl]-2-phenyl-ethyl}-amide; 5-chloro-1H-indole-2-carboxylic acid ((1S)-{(R)-hydroxy-[methyl-(2-pyridin-2-yl-ethyl)-carbamoyl]-methyl}-2-phenyl-ethyl)-amide; 5-chloro-1H-indole-2-carboxylic acid [(1S)-benzyl-(2R)-hydroxy-3-(4-methyl-piperazin-1-yl)-3-oxo-propyl]-amide hydrochloride; 5-chloro-1H-indole-2-carboxylic acid [(1S)-benzyl-(2R)-hydroxy-3-(3-hydroxy-azetidin-1yl)-3-oxo-propyl]-amide; 5-chloro-1H-indole-2-carboxylic acid ((1S)-benzyl-(2R)-hydroxy-3-isoxazolidin-2-yl-3-oxo-propyl)-amide; 5-chloro-1H-indole-2-carboxylic acid ((1S)-benzyl-(2R)-hydroxy-3-[1,2]oxazinan-2-yl-3-oxo-propyl)-amide; 5-chloro-1H-indole-2-carboxylic acid [(1S)-benzyl-(2R)-hydroxy-3-((3S)-hydroxy-pyrrolidin-1yl)-3-oxo-propyl]-amide; 5-chloro-1H-indole-2-carboxylic acid [(1S)-benzyl-3-((3S,4S)-dihydroxy-pyrrolidin-1-yl)-(2R)-hydroxy-3-oxo-propyl]-amide; 5-chloro-1H-indole-2-carboxylic acid [(1S)-benzyl-3-(cis-3,4-dihydroxy-pyrrolidin-1-yl)-(2R)-hydroxy-3-oxo-propyl]-amide; 5-chloro-1H-indole-2-carboxylic acid ((1S)-benzyl-(2R)-hydroxy-3-morpholin-4-yl-3-oxo-propyl)-amide; 5-chloro-1H-indole-2-carboxylic acid [(1S)-benzyl-2-(3-hydroxyimino-pyrrolidin-1yl)-2-oxo-ethyl]-amide; 5-chloro-1H-indole-2-carboxylic acid [2-(cis-3,4-dihydroxy-pyrrolidin-1yl)-2-oxo-ethyl]-amide; 5-chloro-1H-indole-2-carboxylic acid [2-((3S,4S)-dihydroxy-pyrrolidin-1yl)-2-oxo-ethyl]-amide; 5-chloro-1H-indole-2-carboxylic acid [(1S)-benzyl-2-(cis-3,4-dihydroxy-pyrrolidin-1-yl)-2-oxo-ethyl]-amide; 5-chloro-1H-indole-2-carboxylic acid [2-(1,1-dioxo-thiazolidin-3-yl)-2-oxo-ethyl]-amide; 5-chloro-1H-indole-2-carboxylic acid (2-oxo-2-thiazolidin-3-yl-ethyl)-amide, 5-chloro-1H-indole-2-carboxylic acid [(1S)-(4-fluoro-benzyl)-2-(4-hydroxy-piperidin-1yl)-2-oxo-ethyl]-amide; 5-chloro-1H-indole-2-carboxylic acid [(1S)-benzyl-2-((3RS)-hydroxy-piperidin-1-yl)-2-oxo-ethyl]-amide; 5-chloro-1H-indole-2-carboxylic acid [2-oxo-2-((1RS)-oxo-1-thiazolidin-3-yl)-ethyl]-amide; 5-chloro-1H-indole-2-carboxylic acid [(1S)-(2-fluoro-benzyl)-2-(4-hydroxy-piperidin-1-yl)-2-oxo-ethyl]-amide; 5-chloro-1H-indole-2-carboxylic acid [(1S)-benzyl-2-((3S,4S)-dihydroxy-pyrrolidin-1-yl)-2-oxo-ethyl]-amide; 5-chloro-1H-indole-2-carboxylic acid [(1S)-benzyl-2-(3-hydroxy-azetidin-1-yl)-2-oxo-ethyl]-amide; 5-chloro-1H-indole-2-carboxylic acid [(1S)-benzyl-2-(3-hydroxyimino-azetidin-1yl)-2-oxo-ethyl]-amide; 5-chloro-1H-indole-2-carboxylic acid [(1S)-benzyl-2-(4-hydroxyimino-piperidin-1-yl)-2-oxo-ethyl]-amide; 5-chloro-1H-indole-2-carboxylic acid [1-benzyl-2-(3-hydroxypyrrolidin-1yl)-2-oxo-ethyl]amide; 5-chloro-1H-indole-2-carboxylic acid [(1S)—((R)-hydroxy-dimethylcarbamoyl-methyl)-2-phenyl-ethyl]-amide; 5-chloro-1H-indole-2-carboxylic acid [(1S)—((R)-hydroxy-(methoxy-methyl-carbamoyl)-methyl)-2-phenyl-ethyl]-amide; 5-chloro-1H-indole-2-carboxylic acid [(1S)-benzyl-3-((3-hydroxy azetidin-1-yl)-(2R)-hydroxy-3-oxopropyl]-amide; 5-chloro-1H-indole-2-carboxylic acid [(1S)—((R)-hydroxy-[methyl-(2-hydroxyethyl)-carbamoyl]-methyl)-2-phenyl-ethyl]-amide; 5-chloro-1H-indole-2-carboxylic acid [(1S)-benzyl-(2R)-hydroxy-3-((3S)-hydroxy-pyrrolidin-1yl)-3-oxopropyl]-amide; 5-chloro-1H-indole-2-carboxylic acid [(1S)-benzyl-(2R)-hydroxy-3-((3S,4S)-dihydroxy-pyrrolidin-1yl)-3-oxopropyl]-amide; 5-chloro-1H-indole-2-carboxylic acid [(1S)-benzyl-3-(cis-3,4-dihydroxy-pyrrolidin-1yl)-(2R)-hydroxy-3-oxopropyl]-amide; 5-chloro-1H-indole-2-carboxylic acid [1-benzyl-2-(3-hydroxypyrrolidin-1yl)-2-oxo-ethyl]-amide; 5-chloro-1H-indole-2-carboxylic acid [(1S)-benzyl-2-(cis-3,4-dihydroxypyrrolidin-1yl)-2-oxo-ethyl]-amide; 5-chloro-1H-indole-2-carboxylic acid [(1S)-(4-fluorobenzyl-2-(4-hydroxy-piperidin-1yl)-2-oxo-ethyl]-amide; 5-chloro-1H-indole-2-carboxylic acid (2-oxo-2-thiazolidin-3-yl-ethyl)-amide; 5-chloro-1H-indole-2-carboxylic acid [(1S)-benzyl-2-(3-hydroxy-azetidin-1yl)-2-oxo-ethyl]-amide; 5-chloro-1H-indole-2-carboxylic acid [(1S)-benzyl-2-(3-hydroxyimino-azetidin-1yl)-2-oxo-ethyl]-amide; 5-chloro-1H-indole-2-carboxylic acid [(1S)-benzyl-2-((3S,4S)-dihydroxy-pyrrolidin-1-yl)-2-oxo-ethyl]-amide; 3-isopropyl-4-(2-chlorophenyl)-1,4-dihydro-1-ethyl-2-methylpyridine; and the pharmaceutically acceptable salts thereof.

Any glycogen phosphorylase inhibitor may be used in combination with an extract formulation of the present invention. Glycogen phosphorylase inhibition is readily determined by those skilled in the art according to standard assays. A variety of glycogen phosphorylase inhibitors are described above, however, other glycogen phosphorylase inhibitors will be known to those skilled in the art. The following documents also disclose glycogen phosphorylase inhibitors that can be used in the present invention: U.S. Pat. No. 5,952,263, U.S. Pat. No. 5,998,463, WO 95/24391, WO 97/09040, WO 98/40353, WO 98/50359, WO 97/31901, and EP 884050.

In a preferred aspect, the present invention relates to a composition according to the present invention (as defined above), wherein

preferably one, a plurality or all of the glycogen phosphorylase (systematic name: (1→4)-α-D-glucan:phosphate α-D-glucosyltransferase; enzyme classification EC 2.4.1.1) inhibitors are selected from the group (c) consisting of 1,4-dideoxy-1,4-imino-D-arabinitol, isofagomine, and fagomine,

and/or

preferably one, a plurality or all of the antidiabetic active compounds are selected from the group (d) consisting of groups (d-i) and (d-ii) (the corresponding CAS-numbers are indicated in brackets)

(d-i) Tolbutamide (64-77-7), Chlorpropamide (94-20-2), Glyhexamide (451-71-8), Glyoctamide (1038-59-1), Pterostilbene (537-42-8), D-Carnitine (541-14-0), Metformin (657-24-9), Metformin hydrochloride (1115-70-4), Buformin (692-13-7), Phenformin, Acetohexamide (968-81-0), Glimepiride (93479-97-1), Heptolamide (1034-82-8), Tolazamide (1156-19-0), Glymidine sodium (3459-20-9), Glyparamide (5581-42-0), Tolpyrramide (5588-38-5), Butoxamine hydrochloride (5696-15-1), Glyburide (Glibenclamide; 10238-21-8), D-Pinitol (10284-63-6), Glucagon (16941-32-5), Glicetanile sodium (24428-71-5), Glibornuride (26944-48-9), Glipizide (29094-61-9), Gliflumide (35273-88-2), Gliamilide (51876-98-3), Etoformin hydrochloride (53597-26-5), (+)-3-Chlorostyrene oxide (62600-71-9), Pirogliride (62625-18-7), Pirogliride tartrate (62625-19-8), Methyl palmoxirate (69207-52-9), Ciglitazone (74772-77-3), Linogliride (75358-37-1), Linogliride fumarate (78782-47-5), Meglitinide, Palmoxirate sodium (79069-97-9), 3,3,14,14-Tetramethylhexanedecanedioic acid (87272-20-6), Troglitazone (97322-87-7), Seglitide acetate (99248-33-6), Nateglinide (105816-04-4), Englitazone sodium (109229-57-4), Zopolrestat (110703-94-1), Pioglitazone hydrochloride (112529-15-4), Amlintide (122384-88-7), Repaglinide (135062-02-1), Exenatide (141758-74-9), Pramlintide (151126-32-8), Bexarotene (153559-49-0), Rosiglitazone, Rosiglitazone maleate (155141-29-0), Netoglitazone (161600-01-7), Pramlintide acetate (196078-30-5), Liraglutide (204656-20-2), Vildagliptin (274901-16-5), Oxeglitazar (280585-34-4), Arimoclomol (289893-25-0), Solabegron hydrochloride (451470-34-1), Mecasermin rinfabate (478166-15-3), Metformin-Glipizide mixture (614753-49-0), Sitagliptin phosphate (654671-78-0),

(d-ii) extracts, preferably in dried form, preferably aqueous, alcoholic or aqueous alcoholic extracts in dried form, of vegetal organisms selected from the group consisting of (preferably leaves and/or roots of) Artemisia afra, (preferably leaves, stem and/or roots of) Brachylaena discolor, (preferably leaves of) Brachylaena elliptica, (preferably roots of) Bulbine natalenis, (preferably roots of) Bulbine frutescens, (preferably roots of) Cannabis sativa, (preferably leaves, stem and/or roots of) Catha edulis, (preferably leaves and/or twigs of) Catharanthus roseus, (preferably leaves and/or twigs of) Chilianthus olearaceus, Chironia baccifera, (preferably leaves of) Cissampelos capensis, (preferably leaves of) Conyza scabrida, (preferably leaves of) Elytropappus rhinocerotis, (preferably roots of) Galium tomentosum, (preferably leaves and/or roots of) Henrichrysum nudifolium, Herichrysum odoratissimum, Herichrysum petiolare, (preferably leaves and/or roots of) Heteromorphica arborescens, (preferably tubers of) Hypoxis colchicifolia, (preferably tubers of) Hypoxis hemerocallidea, (preferably leaves and/or flowers of) Leonotis leonurus, (preferably stem and/or flowers of) Momordica balsamica, Momordica foetida, (preferably leaves of) Petroselenium crispum, (preferably leaves of) Ricinus communis, (preferably leaves of) Ruta graveolens, (preferably stem, bark and/or roots of) Sclerocarya birrea, (preferably leaves of) Sutherlandia frutescens, (preferably leaves, stem and/or roots of) Vinca major, (preferably leaves, twigs and/or roots of) Vernonia oligocephala, and (preferably leaves of) Vernonia amygdalina.

An extract formulation according to the present invention or produced according to a method of the present invention may also be administered in combination with one or more further anti-obesity agents and/or one or more cholesterol-lowering agents.

Such anti-obesity and/or cholesterol lowering agents preferably are selected from atorvastatin, cerivastatin, fluvastatin, lovastatin, pravastatin, rosuvastatin, simvastatin, sibutramine, diethylpropion, phendimetrazine, phentermine, fenfluramine, dexfenfluramine, bromocriptine, orlistat, ephedrine, leptin, phenylpropanolamine, pseudoephedrine, {4-[2-(2-[6-aminopyridin-3-yl]-2(R)-hydroxyethylamino)ethoxy]phenyl}acetic acid, {4-[2-(2-[6-aminopyridin-3-yl]-2(R)-hydroxyethylamino)ethoxy]phenyl}benzoic acid, {4-[2-(2-[6-aminopyridin-3-yl]-2(R)-hydroxyethylamino)ethoxy]phenyl}propionic acid, and {(4-[2-(2-[6-aminopyridin-3-yl]-2(R)-hydroxyethylamino)ethoxy]phenoxy}acetic acid.

Other beneficial drugs or active agents may be administered in combination with an extract formulation according to the present invention are, e.g. psychoactive agents, agents that help in the treatment of addictive behaviour, e.g. nicotine addiction, or the like, especially in so far as they help to support the prophylaxis or treatment according to the invention intended.

A preferred composition according to the present invention additionally comprises one, two or more further ingredients selected from the group consisting of: preservatives, antimicrobial agents, antiinflammatory agents, antiirritants, antioxidants, chelating agents, moisture regulators, UV filters, fatty oils, fats, saturated fatty acids, mono- or polyunsaturated fatty acids, alpha-hydroxy acids, polyhydroxy-fatty acids, abrasives, binders, thickeners, buffers, dyestuffs, colorants, pigments, film-forming agents, physiological warming agents, physiological cooling agents, emulsifiers, surfactants, detergents, extracts of algae or microalgae, vitamins and electrolytes.

A pharmaceutical or nutraceutical composition according to the present invention can be prepared in various forms, such as granules, tablets, pills, pellets, syrups, solutions, dispersions, emulsions, capsules, suspensions, and the like. Pharmaceutical grade or food grade organic or inorganic carriers and/or diluents suitable for oral use can be used to formulate compositions containing an extract formulation according to the present invention. Diluents known in the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. The compositions may also include one or more of the following: carrier proteins such as serum albumin; buffers; fillers such as microcrystalline cellulose, lactose, corn and other starches; binding agents; sweeteners and other flavouring agents; coloring agents; and polyethylene glycol. Those additives are well known in the art.

In another aspect, the a preferred composition according to the present invention (as defined hereinbefore) comprises an effective amount of an extract formulation obtained according to a method of the present invention (as defined herein) or an extract formulation as defined herein (in each case preferably in a preferred or particularly preferred embodiment), and one or more additional physiologically acceptable carriers, diluents or excipients.

A preferred composition according to the present invention is in a form selected from the group consisting of orally consumable sprays, aerosols, solutions, syrups, dispersions, suspensions, microemulsions, nanoemulsions, o/w-emulsions, w/o-emulsions, and multiple emulsions, granules, tablets, pills, capsules, pellets, and powders.

In another aspect, the present invention relates to a composition according to the present invention (as defined hereinbefore), wherein said composition
comprises one or more additional physiologically acceptable and orally consumable carriers, diluents or excipients,

and/or
is in orally consumable form selected from the group consisting of granules, tablets, pills, capsules, pellets, syrups, powders, emulsions, and dispersions.

In a preferred embodiment, the compositions are preferably formulated in a unit dosage form. The term “unit dosage form” refers to physically discrete units suitable as unitary dosages for human subjects and other mammals, each unit containing a predetermined quantity of an extract formulation according to the present invention to produce the desired therapeutic effect, in association with a suitable pharmaceutical carrier.

Preferably, a composition according to the present invention is formulated in a unit dosage form, preferably selected from the group consisting of granules, tablets, pills, capsules, pellets, and powders, wherein each unit dosage form preferably contains 10 mg to 2000 mg, and preferably from 50 mg to 1000 mg, more preferably from 100 mg to 750 mg of an extract formulation according to the present invention.

Preferably, the total amount of extract formulations according to the present invention administered per subject per day is in the range of 0.5 g to 100 g, more preferably from 0.75 g to 50 g, even more preferably from 1 g to 30 g, particularly preferably from 2 g to 25 g.

In another aspect, the present invention relates to a composition according to the present invention, preferably in one of the preferred embodiments mentioned hereinbefore, for use in a therapeutic or prophylactic method
for treating a disease attendant on hyperglycemia or of a carbohydrate metabolic disorder, preferably prediabetes, obesity, hyperlipemia, arteriosclerosis, arteriolosclerosis, atherosclerosis, and/or diabetes mellitus, in particular type 2 diabetes,

and/or
for treating metabolic syndrome,

and/or
for reducing the degradation of ingested carbohydrates, particularly of one or more polysaccharides, preferably polysaccharides comprising ten or more glucose units, particularly preferably comprising ten or more α-D-glucose units, especially comprising amylose and/or amylopectin,

and/or
for lowering the blood sugar level and/or preventing a high blood sugar level, in particular lowering postprandial blood glucose concentration, preferably in a mammal, especially in a human being,

and/or
for treating or preventing postprandial hyperglycemia.

Where “use” is mentioned in the context or the present invention, this especially refers to one or more of the following embodiments of the invention which can be inserted wherever use is mentioned:

(1) An extract formulation according to the present invention for use in therapeutic (including prophylactic) treatment of a disease attendant on hyperglycemia or of a carbohydrate metabolic disorder, preferably prediabetes, obesity, hyperlipemia, arteriosclerosis, arteriolosclerosis, atherosclerosis, diabetes, postprandial hyperglycemia, and/or treatment of metabolic syndrome, particularly of a mammal, especially a human.

(2) A pharmaceutical or nutraceutical composition comprising an extract formulation according to the present invention as active ingredient together with a pharmaceutically acceptable diluent or carrier, especially for use in the therapeutic and/or prophylactic treatment mentioned under (1).

(2-a) A pharmaceutical or nutraceutical composition for the treatment as mentioned under (1) comprising an extract formulation according to the present invention, and a pharmaceutically acceptable diluent or carrier, as active ingredient supplement to a food.

(3) A functional food comprising an extract formulation according to the present invention, as active ingredient for the treatment as mentioned under (1).

(4) A method for the treatment as mentioned under (1), especially prediabetes, diabetes, postprandial hyperglycemia, atherosclerosis, obesity (adiposity) and/or treatment of metabolic syndrome, in a subject in need of such treatment, comprising administering a pharmaceutically or nutraceutically effective amount of an extract formulation according to the present invention as active ingredient, especially to an individual in need thereof.

(5) The use of an extract formulation according to the present invention as active ingredient for the manufacture of a medicament or nutraceutical or food supplement for the treatment mentioned under (1).

(6) A method or use as defined under (4), comprising co-administration, e.g. concomitantly or in sequence, of a therapeutically effective amount of an extract formulation according to the present invention as active ingredient and a different pharmaceutically active compound and/or a pharmaceutically acceptable salt thereof, said different pharmaceutically active compound and/or salt thereof being especially for use in the treatment as mentioned under (1).

(7) A combination product comprising a therapeutically effective amount of an extract formulation according to the present invention as active ingredient, and a different pharmaceutically active compound and/or a pharmaceutically acceptable salt thereof, said pharmaceutically active compound being especially for use or of use in the treatment mentioned under (1).

The skilled person in the art is familiar with the determining the inhibition of alpha-amylase activity. By way of example, the following publication may be cited in this context: Kasabri V. et al.; In vitro and in vivo acute antihyperglycemic effects of five selected indigenous plants from Jordan used in traditional medicine; Journal of Ethnopharmacology 2011, 133 (2), 888-896.

The compositions according to the present invention may be sterilized and/or may contain carrier materials or adjuvants such as preservatives, stabilizers, binders, disintegrants, wetting agents, skin or mucous membrane penetration enhancers, emulsifiers, salts for varying the osmotic pressure and/or buffers, or other ingredients known in the art.

By physiologically, preferably pharmaceutically and/or nutraceutically, acceptable it is meant that the carrier, diluent or excipient is compatible with the other ingredients of the formulation or composition and not being deleterious to the recipient thereof.

The present compositions may be prepared by known procedures using well known and readily available further ingredients. In making the compositions of the present invention, the extract formulation according to the present invention (as the active ingredient or one of the active ingredients) will usually be admixed with a carrier, or diluted by a carrier, or enclosed within a carrier which may be in the form of a capsule, sachet, paper or other container. When the carrier serves as a diluent, it may be a solid, semi-solid or liquid material which acts as a vehicle, excipient or medium for an extract formulation according to the present invention. The compositions according to the present invention can be in the form of tablets, pills, powders, lozenges, sachets, cachets, elixirs, suspensions, emulsions, solutions, syrups, aerosols, (as a solid or in a liquid medium), ointments, soft and hard gelatin capsules, suppositories, sterile injectable solutions, sterile packaged powders, and the like.

The compositions may additionally include lubricating agents, wetting agents, sweetening agents, flavoring agents, and the like. The compositions of the invention may be formulated so as to provide quick, sustained or delayed release after administration to the patient by employing procedures well known in the art.

In another aspect, the invention relates to the use of an extract formulation according to the present invention or produced according to a method of the present invention (as defined above) in the reduction of glucose uptake content, thereby resulting in body weight management, in particular body weight reduction.

In certain preferred aspects of the present invention, general, preferred or particularly preferred definitions given in the context of the present invention are combined with other preferred or particularly preferred definitions in the context of the present invention.

In certain preferred aspects of the present invention, general, preferred or particularly preferred definitions given in the context of the present invention are combined with preferred or particularly preferred embodiments of the present invention.

The (particularly) preferred aspects and embodiments mentioned hereinbefore or hereinafter relating to extract formulations according to the present invention or produced according to a method of the present invention or compositions according to the present invention also apply to (particularly) preferred aspects and embodiments, uses and methods in accordance with the present invention.

A preferred composition according to the present invention is orally administered 1 second to 60 minutes, preferably 2 to 50 minutes, more preferably 5 to 45 minutes, most preferably 15 to 40 minutes, before food uptake, or during food uptake (i.e. a foodstuff, a food composition, a nutritional product, a meal, or the like).

Preferably, said food uptake is an uptake of food comprising one or more carbohydrates, particularly one or more polysaccharides, preferably polysaccharides comprising ten or more glucose units, particularly preferably comprising ten or more α-D-glucose units, especially comprising amylose and/or amylopectin.

A preferred composition according to the present invention is a pharmaceutical composition, a nutraceutical composition, a nutritional supplement, a functional food, a functional food product, a foodsceutical, a medicinal food, a food composition, or a food supplement.

The present invention also relates to a method for the therapeutic or prophylactic
treatment of a disease attendant on hyperglycemia or of a carbohydrate metabolic disorder, preferably prediabetes, obesity, hyperlipemia, arteriosclerosis, arteriolosclerosis, atherosclerosis, and/or diabetes mellitus, in particular type 2 diabetes,

and/or
treatment of metabolic syndrome,

and/or
treatment or prevention of postprandial hyperglycemia,

and/or
controlling, preferably lowering, glycemia, preferably in a mammal, especially in a human being,

comprising the step of orally administering an effective amount of an extract formulation obtained according to a method according to the present invention as defined hereinbefore, an extract formulation according to the present invention as defined hereinbefore, or a composition according to the present invention as defined hereinbefore,

said effective amount preferably being sufficient to reduce alpha-amylase activity in vitro, preferably to reduce (particularly porcine pancreatic) alpha-amylase activity in vitro by 10% or more, preferably by 20% or more, more preferably by 30% or more, particularly preferably by 40% or more, and most preferably by 50% or more,

wherein said extract formulation or said composition is preferably orally administered 1 second to 60 minutes, preferably 2 to 50 minutes, more preferably 5 to 45 minutes, most preferably 15 to 40 minutes, before food uptake, or during food uptake, in particular of food comprising one or more carbohydrates, particularly one or more polysaccharides, preferably polysaccharides comprising ten or more glucose units, particularly preferably comprising ten or more α-D-glucose units, especially comprising amylose and/or amylopectin.

In particular, the present invention relates to an extract formulation obtained according to a method as defined hereinbefore, an extract formulation according to the present invention (as defined above), or a composition as defined above (in each case preferably in a preferred or particularly preferred embodiments as indicated above) for controlling the body weight and/or for use in a therapeutic method for preventing and/or treating obesity.

Substances and auxiliaries which a composition according to the invention containing an extract formulation according to the present invention or produced according to a method of the present invention may additionally contain are preferably selected from the following group:

preservatives, in particular those described in US 2006/0089413, antimicrobial agents, such as e.g. antibacterial agents or agents to treat yeast and mold, in particular those described in WO 2005/123101, antiirritants (antiinflammatory agents, irritation-preventing agents, irritation-inhibiting agents), in particular those described in WO 2007/042472 and US 2006/0089413, antioxidants, in particular those described in WO 2005/123101, carrier materials, in particular those described in WO 2005/123101, chelating agents, in particular those described in WO 2005/123101, moisture regulators (moisture-donating agents, moisturizing substance, moisture-retaining substances), in particular those described in WO 2005/123101, osmolytes, in particular those described in WO 2005/123101, skin-cooling agents, in particular those described in WO 2005/123101, skin warming agents, in particular those described in WO 2005/123101, UV-absorbing agents, in particular those described in WO 2005/123101, UV filters, in particular those described in WO 2005/123101, further plant parts, plant extracts, in particular those described in WO 2005/123101, vitamins, in particular those described in WO 2005/123101, emulsifiers, in particular those described in WO 2005/123101, gelling agents, in particular those described in WO 2005/123101, oils in particular those described in WO 2005/123101, waxes in particular those described in WO 2005/123101, fats in particular those described in WO 2005/123101, phospholipids, in particular those described in WO 2005/123101, saturated fatty acids and mono- or polyunsaturated fatty acids and alpha-hydroxy acids and polyhydroxy-fatty acids and esters of saturated and/or unsaturated branched and/or unbranched alkane carboxylic acids, in particular those described in WO 2005/123101, surface-active substances (surfactants) in particular those described in WO 2005/123101, dyestuffs and colorants and pigments, in particular those described in WO 2005/123101, aroma chemicals and flavors, in particular those described in S. Arctander, Perfume and Flavor Chemicals, private publishing house, Montclair, N.J., 1969 and Surburg, Panten, Common Fragrance and Flavor Materials, 5th Edition, Wiley-VCH, Weinheim 2006, alcohols and polyols, in particular those described in WO 2005/123101, organic solvents, in particular those described in WO 2005/123101, silicones and silicone oils and silicone derivatives in particular those described in WO 2008/046676, virucides, abrasives, astringents, antiseptic agents, antistatics, binders, buffers, cell stimulants, cleansing agents, softeners, enzymes, essential oils, in particular those described in US 2008/0070825, fibres, film-forming agents, fixatives, foam stabilizers, substances for preventing foaming, foam boosters, gel-forming agents, bleaching agents, optically brightening agents, lubricants, opacifying agents, plasticizing agents, covering agents, gloss agents, polymers in particular those described in WO 2008/046676, powders, peptides, skin-healing agents, stabilizers, suspending agents, thickeners, yeast extracts, algae or microalgae extracts, animal extracts, liquefiers, and electrolytes.

Preferred liquid carrier substances, which may be a component of a composition according to the invention are selected from the group consisting of glycerol, 1,2-propylene glycol, 1,2-butylene glycol, 1,3-butylene glycol, 1,2-pentanediol, 1,2-hexanediol, ethanol, water and mixtures of two or more of said liquid carrier materials with water.

Further additional beneficial agents which may be part of a composition according to the present invention are preferably are selected from the group consisting of sodium lactate, lecithin, lycopene, phytosterols, amino acids, vitamin E and derivatives (preferably tocopherol, tocopheryl acetate), vitamin C and derivatives (ascorbic acid, ascorbyl palmitate), alpha-hydroxy acids (preferably citric acid, lactic acid, malic acid) and derivatives thereof, galactose, fructose, mannose, beta-glucans, in particular 1,3-1,4-beta-glucan (preferably from oats), alpha-hydroxy-fatty acids, triterpenic acids, such as betulic acid or ursolic acid, and algae extracts.

The present invention is further explained by the following examples. The specific examples which follow illustrate the methods in which the extract formulations and compositions of the present invention may be prepared and used.

### EXAMPLES

### Example 1

### Extract Formulations

Roots and leaves from Rhodamnia cinerea (collected in Malaysia) were used in the following experiments.

Preparation of Extract Formulations by Using Water or Aqueous Ethanolic Extractants

200 g air dried plant material, either roots (material code BTP-00166) or leaves (material code BTP-00167), were ground into a powder using a laboratory mill. Each 50 g aliquots of the powdered plant material were used to prepare hot water extracts (see example 1.1) and aqueous ethanolic extracts (see example 1.2), prepared by extraction with solvents consisting of ethanol and water. The ethanol proportions in the solvent used in the respective extraction were 30, 70 and 90 vol. %, respectively.

### Example 1.1

### Hot Water Extraction

50 g of the respective powdered plant materials (i.e. roots or leaves) were each extracted separately with 700 ml water under reflux for 1 hour. Then, the water extract was separated from the remaining plant materials by filtration. Subsequently, the extract obtained was concentrated under reduced pressure (rotary evaporator, max. water bath temperature 40° C.), and finally dried by lyophilisation. The yields for the resulting dried extract formulations are given in Table 1 below.

### Example 1.2

### Organic Extractions (Mixtures of Water and Ethanol)

From roots and leaves from Rhodamnia cinerea each three extract formulations were prepared with ethanol/water mixtures.

50 g of the respective powdered plant material (either roots or leaves) were each extracted separately with 300 ml of the respective ethanol/water mixture (ethanol:water=30:70, 70:30, and 90:10 (v/v)) by subjecting the mixture to ultrasonication for 30 min. at a maximum temperature of 40° C. The resulting suspensions were filtered and the residual plant material was extracted a second time under the same conditions. The combined filtrates were concentrated under reduced pressure at a maximum temperature of 40° C., thereby removing essentially all the ethanol and a major part of the water. Finally, the material was dried by lyophilisation. The yields for the thus obtained extract formulations are given in Table 1.

TABLE 1
Rhodamnia cinerea extract formulations: yields after lyophilisation

| Rhodamnia cinerea | | | |
|---|---|---|---|
| plant part used | Material Code | Extractant | Yield (mg) |
| Roots | BTP-00166-04 | H₂O | 3794.5 |
| | BTP-00166-05 | 30 vol. % EtOH | 1993.5 |
| | BTP-00166-06 | 70 vol. % EtOH | 1664.5 |
| | BTP-00166-07 | 90 vol. % EtOH | 1707.2 |
| Leaves | BTP-00167-14 | H₂O | 7295.3 |
| | BTP-00167-15 | 30 vol. % EtOH | 5460.3 |
| | BTP-00167-16 | 70 vol. % EtOH | 3429.3 |
| | BTP-00167-17 | 90 vol. % EtOH | 5525.4 |

### Example 2

### In Vitro Alpha Amylase Inhibition Testing

The activity of porcine pancreatic amylase with and without Rhodamnia cinerea extract formulation was determined in a colorimetric assay using Starch Azure as substrate solution. Porcine pancreatic α-amylase is an endo-type amylase that catalyzes the hydrolysis of α-(1,4) glucosidic bonds in amylose and amylopectin.

The reference standard, alpha-amylase inhibitor type I from Triticum aestivum (wheat seed; product A1520 obtained from Sigma-Aldrich), was run as a positive control to ensure the validity of the results obtained.

Assay Set Up and Procedure:

The enzymatic reaction was performed in a NaH₂PO₄ buffer (20 mM NaH₂PO₄, 50 mM NaCl, pH 7). The enzyme (15 U/ml) was preincubated with the respective extract formulation, the positive control and the enzyme alone for 10 minutes at room temperature, followed by the addition of the substrate starch azure at a final concentration of 1.75% (w/v). This solution was incubated for 30 minutes at 37° C. and the enzymatic reaction was stopped afterwards by the addition of acetic acid (2M final concentration). After a centrifugation for 1 minute at 13000 rpm the absorption of the supernatant was determined at 595 nm. The inhibition of was calculated based on the substrate turnover in relation to the uninhibited enzyme.

Results:

The inhibition of α-amylase was tested at concentrations of 50, 12.5, 3.125, and 0.78 μg/ml (n=3). An inhibitor type I obtained from Triticum aestivum served as positive control resulting in an 88.9% inhibition using 22.5 units/ml. All extracts from leaves and roots, either the hot water or the aqueous ethanolic extracts showed a dose-dependant inhibition of alpha-amylase, see Table 2.

TABLE 2
In vitro alpha-amylase inhibition
measurements at different concentrations

| | | In vitro inhibition of alpha-amylase at | | | |
|---|---|---|---|---|---|
| Rhodamnia | Material | 50 | 12.5 | 3.13 | 0.78 |
| cinerea | Code | μg/ml | μg/ml | μg/ml | μg/ml |
| Roots | BTP-00166-04 | 99.0% | 91.4% | 29.3% | 0.3% |
| | BTP-00166-05 | 97.2% | 96.1% | 26.4% | 17.7% |
| | BTP-00166-06 | 96.3% | 92.4% | 26.8% | 0.0% |
| | BTP-00166-07 | 100.0% | 53.2% | 4.9% | 3.5% |
| Leaves | BTP-00167-14 | 97.1% | 93.1% | 32.8% | 12.6% |
| | BTP-00167-15 | 97.6% | 93.9% | 34.1% | 8.6% |
| | BTP-00167-16 | 97.9% | 90.5% | 35.6% | 24.5% |
| | BTP-00167-17 | 99.4% | 93.6% | 33.6% | 17.2% |

### Example 3

### Acute Starch Tolerance Test (STT)—In Vivo Model for Evaluation of Effect of Alpha-Amylase Inhibitors on Glycemia

An acute Starch Tolerance Test (STT) was performed by the administration by oral gavage of a 7.5% purified wheat starch solution at 1.5 g/kg of body weight to normal male Wistar rats. The effect of a concomitantly administered hot water extract formulation from Rhodamnia cinerea leaves (BTP-00167-14; ref. example 1.2) on the glycemic index was measured at different doses orally administered (50 and 200 mg/kg of body weight).

The extract formulation BTP-00167-14 was administered by oral route concomitantly with wheat starch solutions. Glycemia was measured before and 15, 30, 60, 90, and 120 min after mixture administration. The positive control group received acarbose at 5 mg/kg of body weight together with the starch.

The actual volume administered to each rat was calculated and adjusted based on the most recent body weight of each animal. STT onset was between 12:00 p.m. (noon) and 1:00 p.m. on animals fasted overnight.

Blood samples (one drop) were collected via the tail vein for glucose determination using a hand-held glucometer before and 15, 30, 60, 90, and 120 min after starch administration.

Seven-week old rats weighing around 135 g were used for the experiment. One day before the test, rats were randomly assigned to the different experimental groups.

| | Animals | | Dosage of |
|---|---|---|---|
| Group | per group | Test item | actives |
| 1 | 10 | starch (control) | |
| 2 | 10 | starch + Acarbose* (positive control) | 5 mg/kg |
| 3 | 10 | starch + BTP-00167-14 | 50 mg/kg |
| 4 | 10 | starch + BTP-00167-14 | 200 mg/kg |

*obtained from Sigma Aldrich (product A8980)

Results:

The hot water extract formulation of Rhodamnia cinerea (BTP-00167-14) reduced the glycemic response to starch by 17.9% at the dose of 50 mg/kg (area under the curve 0-120; p<0.001 and by 38.5% at the dose of 200 mg/kg of body weight (area under the curve 0-120; p<0.001). Regarding time points independently, this effect is statistically significant at 15, 30, and 60 min and also at 90 min for the highest dose of 200 mg/kg of body weight.

Delta glycemia values were calculated by subtraction of the pre-STT glycemia values (Time 0). Values represent average (i.e. mean) values.

TABLE 3
Effects of the hot water extract formulation from leaves
of Rhodamnia cinerea (BTP-00167-14) in the acute Starch
Tolerance Test (STT) compared to acarbose in rats:

| | Dosage | Glycemia [mg/dL] | | | | | |
|---|---|---|---|---|---|---|---|
| Material | [mg/kg] | 0 min | 15 min | 30 min | 60 min | 90 min | 120 min |
| Starch (control) | | 55.8 | 158.7 | 173.9 | 157.9 | 126.7 | 101.4 |
| Acrabose | 5 | 55.0 | 72.4** | 83.7** | 71.7** | 66.0** | 61.9** |
| (positive control) | | | | | | | |
| BTP-00167-14 | 50 | 54.0 | 128.0** | 143.4** | 134.8** | 120.3 | 103.1 |
| | 200 | 55.4 | 107.5** | 124.5** | 113.5** | 105.2** | 96.9 |

**p < 0.001

1. Method for producing an extract formulation of Rhodamnia cinerea comprising or consisting of the following steps:
(i) providing plant material from Rhodamnia cinerea,
(i-a) optionally drying the plant material provided in step (i),
(ii) extracting the plant material provided in step (i) or (i-a) with an extractant essentially consisting or consisting of water or a mixture essentially consisting or consisting of an alcohol having 1 to 3 carbon atoms and water,
(iii) optionally mixing the extract obtained in step (ii) with one or more solid carrier substances, preferably one or more solid carrier substances selected from the group consisting of maltodextrins, silica, talc, lactose, sorbitol, mannitol, dextrose, sucrose, starches, gums, orally consumable calcium salts, orally consumable stearate salts, alginates, tragacanth, gelatins, cellulose and cellulose derivatives, polyvinylpyrrolidones, and propylhydroxybenzoates,
(iv) drying the extract obtained in step (ii) or the mixture obtained in step (iii), preferably by spray-drying or freeze-drying, preferably drying until the total amount of water and alcohols having 1 to 3 carbon atoms is below 15 wt. %, preferably below 10 wt. %, more preferably below 5 wt. %, most preferably below 3 wt. %, based on the total weight of the extract formulation.

2. Method according to claim 1, wherein the plant material provided in step (i) comprises leaves and/or roots of Rhodamnia cinerea.

3. Method according to claim 1, wherein in step (ii) the extraction is performed with an extractant
essentially consisting or consisting of water, or
a mixture essentially consisting or consisting of an alcohol having 1 to 3 carbon atoms and water, preferably a mixture of ethanol and water, wherein the total volume ratio (v/v) of said alcohol:water is in the range of 1:20 to 25:1, preferably in the range of 1:12 to 12:1, more preferably in the range of 1:6 to 10:1, even more preferably in the range of 1:5 to 5:1, particularly preferably in the range of 1:3 to 3:1, and most preferably in the range of 2:5 to 5:2.

4. Method according to claim 1, wherein in step (ii) the extraction is performed at a temperature in the range of 40 to 120° C., preferably in the range of 50 to 110° C., more preferably in the range of 60 to 100° C.

5. Method according to claim 1, wherein in step (iii) the extract obtained in step (ii) is mixed with one or more solid carrier substances selected from the group consisting of maltodextrins, silica, talc, lactose, sorbitol, mannitol, dextrose, sucrose, starches, gum acacia, calcium phosphates, calcium silicates, magnesium stearate, alginates, tragacanth, gelatins, amorphous cellulose, microcrystalline cellulose, methyl cellulose, polyvinylpyrrolidones, and propylhydroxybenzoates.

6. Extract formulation in solid form obtained from plant material from Rhodamnia cinerea, preferably obtainable or obtained by a method according to claim 1.

7. Extract formulation obtained according to a method for producing an extract formulation of Rhodamnia cinerea comprising or consisting of the following steps:
(i) providing plant material from Rhodamnia cinerea,
(i-a) optionally drying the plant material provided in step (i),
(ii) extracting the plant material provided in step (i) or (i-a) with an extractant essentially consisting or consisting of water or a mixture essentially consisting or consisting of an alcohol having 1 to 3 carbon atoms and water,
(iii) optionally mixing the extract obtained in step (ii) with one or more solid carrier substances, preferably one or more solid carrier substances selected from the group consisting of maltodextrins, silica, talc, lactose, sorbitol, mannitol, dextrose, sucrose, starches, gums, orally consumable calcium salts, orally consumable stearate salts, alginates, tragacanth, gelatins, cellulose and cellulose derivatives, polyvinylpyrrolidones, and propylhydroxybenzoates,
(iv) drying the extract obtained in step (ii) or the mixture obtained in step (iii), preferably by spray-drying or freeze-drying, preferably drying until the total amount of water and alcohols having 1 to 3 carbon atoms is below 15 wt. %, preferably below 10 wt. %, more preferably below 5 wt. %, most preferably below 3 wt. %, based on the total weight of the extract formulation, or extract formulation thereof for use in a therapeutic or prophylactic method selected from the group consisting of:
for treating a disease attendant on hyperglycemia or of a carbohydrate metabolic disorder, preferably prediabetes, obesity, hyperlipemia, arteriosclerosis, arteriolosclerosis, atherosclerosis, and/or diabetes mellitus, in particular type 2 diabetes, and/or
for treating metabolic syndrome,
for reducing the degradation of ingested carbohydrates, particularly of one or more polysaccharides, preferably polysaccharides comprising ten or more glucose units, particularly preferably comprising ten or more α-D-glucose units, especially comprising amylose and/or amylopectin,
for controlling, preferably lowering, glycemia, preferably in a mammal, especially in a human being,
and
for treating or preventing postprandial hyperglycemia.

8. Use of an extract obtained by extraction of plant material of Rhodamnia cinerea with an extractant essentially consisting or consisting of water or a mixture essentially consisting or consisting of an alcohol having 1 to 3 carbon atoms and water, preferably of an extract formulation obtained by a method for producing an extract formulation of Rhodamnia cinerea comprising or consisting of the following steps:
(i) providing plant material from Rhodamnia cinerea,
(i-a) optionally drying the plant material provided in step (i),
(ii) extracting the plant material provided in step (i) or (i-a) with an extractant essentially consisting or consisting of water or a mixture essentially consisting or consisting of an alcohol having 1 to 3 carbon atoms and water,
(iii) optionally mixing the extract obtained in step (ii) with one or more solid carrier substances, preferably one or more solid carrier substances selected from the group consisting of maltodextrins, silica, talc, lactose, sorbitol, mannitol, dextrose, sucrose, starches, gums, orally consumable calcium salts, orally consumable stearate salts, alginates, tragacanth, gelatins, cellulose and cellulose derivatives, polyvinylpyrrolidones, and propylhydroxybenzoates,
(iv) drying the extract obtained in step (ii) or the mixture obtained in step (iii), preferably by spray-drying or freeze-drying, preferably drying until the total amount of water and alcohols having 1 to 3 carbon atoms is below 15 wt. %, preferably below 10 wt. %, more preferably below 5 wt. %, most preferably below 3 wt. %, based on the total weight of the extract formulation, or extract formulation thereof, selected from the group consisting of
as alpha-amylase inhibitor,
for reducing the activity of mammalian alpha-amylase,
in a food composition, a nutraceutical composition or a food supplement,
and
for the manufacture of a food composition, a nutraceutical composition or a food supplement.

9. Composition, preferably orally administrable composition, comprising an effective amount of an extract formulation as obtained by a method for producing an extract formulation of Rhodamnia cinerea comprising or consisting of the following steps:
(i) providing plant material from Rhodamnia cinerea,
(i-a) optionally drying the plant material provided in step (i),
(ii) extracting the plant material provided in step (i) or (i-a) with an extractant essentially consisting or consisting of water or a mixture essentially consisting or consisting of an alcohol having 1 to 3 carbon atoms and water,
(iii) optionally mixing the extract obtained in step (ii) with one or more solid carrier substances, preferably one or more solid carrier substances selected from the group consisting of maltodextrins, silica, talc, lactose, sorbitol, mannitol, dextrose, sucrose, starches, gums, orally consumable calcium salts, orally consumable stearate salts, alginates, tragacanth, gelatins, cellulose and cellulose derivatives, polyvinylpyrrolidones, and propylhydroxybenzoates,
(iv) drying the extract obtained in step (ii) or the mixture obtained in step (iii), preferably by spray-drying or freeze-drying, preferably drying until the total amount of water and alcohols having 1 to 3 carbon atoms is below 15 wt. %, preferably below 10 wt. %, more preferably below 5 wt. %, most preferably below 3 wt. %, based on the total weight of the extract formulation, or extract formulation thereof, said effective amount selected from the group consisting of and being sufficient
to reduce alpha-amylase activity in vitro, preferably to reduce alpha-amylase activity in vitro by 10% or more, preferably by 20% or more, more preferably by 30% or more, most preferably by 50% or more,
and
to reduce the glycemic response to orally administered wheat starch in an amount of 1.5 g/kg in vivo in rats by 10% or more, preferably by 15% or more, more preferably by 20% or more, measured 30 minutes after oral administration of the wheat starch.

10. Composition according to claim 9, additionally comprising one or more further glycosidase inhibitors, wherein said further glycosidase inhibitors are preferably selected from the group consisting of oligo-1,6-glucosidase inhibitors, alpha-glucosidase inhibitors, amylo-alpha-1,6-glucosidaseinhibitors, sucrose alpha-glucosidase inhibitors, isoamylase inhibitors, lactase inhibitors, and further alpha-amylase inhibitors.

11. Composition according to claim 10, comprising one or more further glycosidase inhibitors, wherein preferably one, a plurality or all of the further glycosidase inhibitors are selected from the group consisting of
acarbose, miglitol, voglibose, camiglibose, pradimicin-Q, saponarin, mahanimbine, gymnemic acids, S-allyl cysteine sulphoxide, nojirimycin, 1-deoxynojirimycin, N-methyl-1-deoxynojirimycin, deoxygalactonojirimycin, emiglitate, adiposines, swainsonine, australine, 2-amino-3,4-dihydroxy-5-methoxybenzoic acid, castanospermine, 6-epicastanospermine, 2,5-dihydroxymethyl-3,4-dihydroxypyrrolidine, salacinol, kotalanol, fustin, fisetin, gallic acid, methyl gallate, 3′,4′,7′-trihydroxyflavone, (−)-3-O-galloylepicatechin, (−)-3-O-galloylcatechin, epicatechin, salbostatin, and the pharmaceutically acceptable salts thereof,
extracts, dried extracts or dried parts of vegetal organisms selected from the group consisting of Aegle marmeloes, Aloe vera, Anacardium occidentale, Artemisia santolina, Asparagas racemosus, Berberis integrimma, Brassica nigra, Camellia sinensis, Cannabis sativa, Cassia auriculata, Cassia fistula, Cichorium intybus, Citrus aurantium, Coccinia indica, Crocus sativa, Cuminum cymirum, Eugenia jambolana, Ficus bengalensis, Ficus carica, Foeniculum vulgare, Glycyrrhiza glabra, Gossypium arboreturn, Holarina antidysentrica, Lawsonia inermis, Nigella sativa, Phyllanthus amarus, Piper nigrum, Punica granatum, Solanum dulcamara, Strychnos potatorum, Terminalia arjuna, Terminalia chebulla, Grifola frondosa, Schizandra chinensis, Gymnea sylvestre, Momordica charantia, Trigonella foenum graecum, Pterocarpus marsupium, Murraya koenigii, Ocimum sanctum, Tinospora cordifolia, Syzygium cumini, Zingiber officinale, Allium sativum, plants of the genus Salacia, plants of the genus Oenothera, plants of the genus Morus, Phyllantus niruri, Smilax officinalis, flex paraguayensis, Tagetes minuta, Solanum diphyllum, Rhus verniciflua, Rumex nepalensis, Olea europaea, Malpighia glabra, Cornus officinalis, Pelvetia wrightii, Syzygium aromaticum, Tamarindus indica, Camellia ptilophylla, Hydrangea paniculata, Rubus phoenicolasius, Chrysanthemum coronarium, Cyclocarya paliurus, Cymbopogon martinii, Castanea crenata,
L-arabinose, L-fucose, D-xylose, L-xylose, D-ribose, D-tagatose, D-ribulose, D-lyxose, D-xylulose,
extracts, preferably dried extracts, of Alstonia scholaris, Piper umbellatum, Tussilago farfara, Terminalia chebula, Bergenia cilata, Grateloupia elliptica, Syagrus romanzoffiana, Fagara tessmannii, Gypsophila oldhamiana, vasicine, vasicinol, piperumbellactams, chebulanin, chebulagic acid, chebulinic acid, β-hydroxykompasinol A, kompasinol A, scirpusin A, scirpusin C, pentahydroxystilbene, curcumin, demethoxycurcumin, bisdemethoxycurcumin, 3b-acetoxy-16b-hydroxybetulinic acid, cyanidin-3-galactoside,
extracts, preferably dried extracts, of Lagerstoemia speciosa, Camellia sinensis, Psidium guajava, Anacardium occidentale, Syzygium zeylanicum, Cleistocalyx operculatus, Horsefieldia amygdalina, Careya arborea, Phyllanthus amarus, Acanthopanax sieboldianum, Ficus bengalensis, Syzygium cumini, Cinnamonum verum, Curcuma longa, Bixa orellana, Murraya koenigii, Tribulus terrestris, Solanum diphyllum, Aesculus turbinate, Callistemon rigidus, plants of the genus Morus, Phaseolus sp., Phaseolus vulgaris, Triticum spp., Pistacia atlantica, Sarcopoterium spinosum, Rheum ribes,
dicaffeoylquinic acids, oleanolic acid, ursolic acid, lupeol, phaseolamin, scirpusin B, piceatannol, trestatins, tendamistat, and AI-3688.

12. Composition according to claim 9, comprising one or more glycogen phosphorylase inhibitors and/or one or more further antidiabetic active compounds, wherein preferably
one, a plurality or all of the glycogen phosphorylase inhibitors are selected from the group (c) consisting of 1,4-dideoxy-1,4-imino-D-arabinitol, isofagomine, and fagomine,
and/or
one, a plurality or all of the antidiabetic active compounds are selected from the group (d) consisting of groups (d-i) and (d-ii)
(d-i) Tolbutamide, Chlorpropamide, Glyhexamide, Glyoctamide, Pterostilbene, D-Carnitine, Metformin, Metformin hydrochloride, Buformin, Phenformin, Acetohexamide, Glimepiride, Heptolamide, Tolazamide, Glymidine sodium, Glyparamide, Tolpyrramide, Butoxamine hydrochloride, Glyburide, D-Pinitol, Glucagon, Glicetanile sodium, Glibornuride, Glipizide, Gliflumide, Gliamilide, Etoformin hydrochloride, (+)-3-Chlorostyrene oxide, Pirogliride, Pirogliride tartrate, Methyl palmoxirate, Ciglitazone, Linogliride, Linogliride fumarate, Meglitinide, Palmoxirate sodium, 3,3,14,14-Tetramethylhexanedecanedioic acid, Troglitazone, Seglitide acetate, Nateglinide, Englitazone sodium, Zopolrestat, Pioglitazone hydrochloride, Amlintide, Repaglinide, Exenatide, Pramlintide, Bexarotene, Rosiglitazone, Rosiglitazone maleate, Netoglitazone, Pramlintide acetate, Liraglutide, Vildagliptin, Oxeglitazar, Arimoclomol, Solabegron hydrochloride, Mecasermin rinfabate, Metformin-Glipizide mixture, Sitagliptin phosphate,
(d-ii) extracts, preferably in dried form, preferably aqueous, alcoholic or aqueous alcoholic extracts in dried form, of vegetal organisms selected from the group consisting of Artemisia afra, Brachylaena discolor, Brachylaena elliptica, Bulbine natalenis, Bulbine frutescens, Cannabis sativa, Catha edulis, Catharanthus roseus, Chilianthus olearaceus, Chironia baccifera, Cissampelos capensis, Conyza scabrida, Elytropappus rhinocerotis, Galium tomentosum, Herichrysum nudifolium, Herichrysum odoratissimum, Herichrysum petiolare, Heteromorphica arborescens, Hypoxis colchicifolia, Hypoxis hemerocallidea, Leonotis leonurus, Momordica balsamica, Momordica foetida, Petroselenium crispum, Ricinus communis, Ruta graveolens, Sclerocarya birrea, Sutherlandia frutescens, Vinca major, Vernonia oligocephala, and Vernonia amygdalina.

13. Composition according to claim 9, wherein said composition
comprises one or more additional physiologically acceptable and orally consumable carriers, diluents or excipients,
and/or
is in orally consumable form selected from the group consisting of granules, tablets, pills, capsules, pellets, syrups, powders, emulsions, and dispersions.

14. Composition according to claim 9 for use in a therapeutic or prophylactic method
for treating a disease attendant on hyperglycemia or of a carbohydrate metabolic disorder, preferably prediabetes, obesity, hyperlipemia, arteriosclerosis, arteriolosclerosis, atherosclerosis, and/or diabetes mellitus, in particular type 2 diabetes,
and/or
for treating metabolic syndrome,
and/or
for reducing the degradation of ingested carbohydrates, particularly of one or more polysaccharides, preferably polysaccharides comprising ten or more glucose units, particularly preferably comprising ten or more α-D-glucose units, especially comprising amylose and/or amylopectin,
and/or
for lowering the blood sugar level and/or preventing a high blood sugar level, in particular lowering postprandial blood glucose concentration, preferably in a mammal, especially in a human being,
and/or
for treating or preventing postprandial hyperglycemia.

15. Composition according to claim 14, wherein said composition is orally administered 1 second to 60 minutes, preferably 2 to 50 minutes, more preferably 5 to 45 minutes, most preferably 15 to 40 minutes, before food uptake, or during food uptake.

16. Composition according to claim 9, wherein the composition is a pharmaceutical composition, a nutraceutical composition, a nutritional supplement, a functional food, a functional food product, a foodsceutical, a medicinal food, a food composition, or a food supplement.

17. The method according to claim 1 further comprising a Method for the therapeutic or prophylactic
treatment of a disease attendant on hyperglycemia or a of carbohydrate metabolic disorder, preferably prediabetes, obesity, hyperlipemia, arteriosclerosis, arteriolosclerosis, atherosclerosis, and/or diabetes mellitus, in particular type 2 diabetes,
and/or
treatment of metabolic syndrome,
and/or
treatment or prevention of postprandial hyperglycemia,
and/or
controlling, preferably lowering, glycemia, preferably in a mammal, especially in a human being,
comprising the step of orally administering an effective amount of an extract formulation obtained according to the method defined in claim 1,
wherein said extract formulation or said composition is preferably orally administered 1 second to 60 minutes, preferably 2 to 50 minutes, more preferably 5 to 45 minutes, most preferably 15 to 40 minutes, before food uptake, or during food uptake, in particular of food comprising one or more carbohydrates, particularly one or more polysaccharides, preferably polysaccharides comprising ten or more glucose units, particularly preferably comprising ten or more α-D-glucose units, especially comprising amylose and/or amylopectin.

18. The method according to claim 6 further comprising a Method for the therapeutic or prophylactic
treatment of a disease attendant on hyperglycemia or a of carbohydrate metabolic disorder, preferably prediabetes, obesity, hyperlipemia, arteriosclerosis, arteriolosclerosis, atherosclerosis, and/or diabetes mellitus, in particular type 2 diabetes,
and/or
treatment of metabolic syndrome,
and/or
treatment or prevention of postprandial hyperglycemia,
and/or
controlling, preferably lowering, glycemia, preferably in a mammal, especially in a human being,
comprising the step of orally administering an effective amount of an extract formulation obtained according to the extract formulation according to claim 6,
wherein said extract formulation or said composition is preferably orally administered 1 second to 60 minutes, preferably 2 to 50 minutes, more preferably 5 to 45 minutes, most preferably 15 to 40 minutes, before food uptake, or during food uptake, in particular of food comprising one or more carbohydrates, particularly one or more polysaccharides, preferably polysaccharides comprising ten or more glucose units, particularly preferably comprising ten or more α-D-glucose units, especially comprising amylose and/or amylopectin.

19. The method according to claim 9 further comprising a Method for the therapeutic or prophylactic
treatment of a disease attendant on hyperglycemia or a of carbohydrate metabolic disorder, preferably prediabetes, obesity, hyperlipemia, arteriosclerosis, arteriolosclerosis, atherosclerosis, and/or diabetes mellitus, in particular type 2 diabetes,
and/or
treatment of metabolic syndrome,
and/or
treatment or prevention of postprandial hyperglycemia,
and/or
controlling, preferably lowering, glycemia, preferably in a mammal, especially in a human being,
comprising the step of orally administering an effective amount of an extract formulation obtained according to the composition defined in claim 9,
wherein said extract formulation or said composition is preferably orally administered 1 second to 60 minutes, preferably 2 to 50 minutes, more preferably 5 to 45 minutes, most preferably 15 to 40 minutes, before food uptake, or during food uptake, in particular of food comprising one or more carbohydrates, particularly one or more polysaccharides, preferably polysaccharides comprising ten or more glucose units, particularly preferably comprising ten or more α-D-glucose units, especially comprising amylose and/or amylopectin.
